(19) United States
(12) Patent Application Publication
(10) Pub. No.: US 2017/0260543 A1
(43) Pub. Date: Sep. 14, 2017

(54) COMPOSITIONS AND METHODS FOR FLUORESCENT GENETIC BAR-CODING IN MAMMALIAN CELLS FOR ENHANCED MULTIPLEXING CAPABILITIES IN FLOW CYTOMETRY

(71) Applicant: SAN DIEGO STATE UNIVERSITY (SDSU), dba SAN DIEGO STATE UNIVERSITY (SDSU) RESEARCH FOUNDATION, San Diego, CA (US)

(72) Inventor: Roland WOLKOWICZ, San Diego, CA (US)

(21) Appl. No.: 15/439,468

(22) Filed: Feb. 22, 2017

Related U.S. Application Data
(63) Continuation of application No. 14/462,488, filed on Aug. 18, 2014, now abandoned.
(60) Provisional application No. 61/867,533, filed on Aug. 19, 2013.

(51) Int. Cl. C12N 15/85; G01N 15/14; G01N 21/64; A01K 67/027

(52) U.S. Cl. CPC C12N 15/85; A01K 67/0275; G01N 15/1456; G01N 21/6428; A01K 2217/052; G01N 2015/1006

(57) ABSTRACT

The invention provides cells or populations of cells, including non-human animals or non-human mammals having these cells, where the cells or populations of cells are stably tagged, uniquely identified and genetically barcoded by one or more detectable, e.g., fluorescent, proteins; and methods of making and using them. In alternative embodiments, the invention provides methods for tagging, uniquely identifying or genetically barcoding a cell, a population of cells, or a culture of cells by stably transferring, transfecting, transducing, infecting or implanting one or more nucleic acids encoding readable or detectable, e.g., fluorescent, moieties into the cells. In alternative embodiments, the nucleic acids are stably inserted into the cells such that the readable or detectable, e.g., fluorescent, genetic barcoding becomes a stable, heritable characteristic of the cell. In alternative embodiments, the invention provides fluorescent barcoded multiplexed cell-based assays using several different fluorescent proteins. The multiplexing power of methods of the invention can be increased by combining both the number of distinct fluorescent proteins and the fluorescence intensity in each channel.

### RELATED APPLICATIONS

This application is a continuation of U.S. patent application Ser. No. (“USSN”) 14/462,488, filed Aug. 18, 2014, which claims priority under 37 CFR §119(e) to U.S. Patent Application Ser. No. (“USSN”) 61/867,533, filed Aug. 19, 2013 (currently pending). The aforementioned application is expressly incorporated herein by reference in its entirety and for all purposes.

### TECHNICAL FIELD

This invention relates to molecular and cellular biology, molecular genetics, and cell assays. In one aspect, the invention is directed to methods for tagging, uniquely identifying or genetically barcoding a cell, a population of cells, or a culture of cells by stably transferring, transfecting, transducing, infecting or implanting one or more nucleic acids encoding readable or detectable, e.g., fluorescent, moieties into the cells. In alternative embodiments, the nucleic acids are stably inserted into the cells such that the readable or detectable, e.g., fluorescent, genetic barcoding becomes a stable, heritable characteristic of the cell. In alternative embodiments, the invention provides fluorescent barcoded multiplexed cell-based assays using several different fluorescent proteins. The multiplexing power of methods of the invention can be increased by combining both the number of distinct fluorescent proteins and the fluorescence intensity in each channel.

### BACKGROUND

Since the isolation and cloning of the green fluorescent protein (GFP) from Aequorea victoria (1), fluorescent proteins have revolutionized all aspects of biomedical research particularly the field of flow cytometry. The expression of these proteins in mammalian cells and others has enabled tracking of individual cells within a large population, enabling the study of cell fate. Moreover, they have been crucial for the study of gene regulation, and their use as tags within fluorescent fusions have dramatically facilitated the investigation of their biological functions and consequences (2). The introduction of retroviral technology that enables protein expression in mammalian cells in a stable manner has extended the advantages of fluorescent proteins (3-7).

The ability to stably express genes in mammalian cells together with the discovery of an increasing number of fluorescent proteins and genetic manipulations of GFP (8), has further enhanced the utility of flow cytometry for cell analysis (9,10). Novel fluorescent proteins with broader absorbance/emission spectra and larger Stokes shifts (11-13) have been introduced in conjunction with additional probes, dyes, and lasers of varying wavelengths (14-18). This has allowed for the analysis of an ever-increasing number of parameters that can theoretically be analyzed concomitantly in the same sample at the same time. However, the multi-parameter aspects of the current instrumentation do not always match the experimental design, nor reflect the appropriate technological capabilities.

Multi-parameter, multifaceted applications are thus made available by flow cytometry and should allow for more complex analysis or utilities than classical detection of gene expression, cell cycle, apoptosis, phosphorylation events, or any general biological question at the single cell level (10,19-24). The introduction of robotics in plate reader systems, together with new imaging and flow cytometry coupled applications, has significantly increased high throughput capabilities in biomedical research (21,25), as demonstrated by novel applications involving, but not restrained to, the use of cell-based assays as a platform for drug screening (22,26-29), as well as multi-parameter analysis of signaling cascades (20,30,31). Typically, the preparation of these samples includes time-consuming protocols and significant amounts of costly reagents including antibodies, beads and/or stains (22,32,33). While many of these procedures can be calibrated in advance to reduce cost and time, such optimization is not always feasible and requires a higher degree of expertise.

A growing number of biological applications in clinical and/or research settings, in parallel to the growing technological capabilities of the available instrumentation (25,34-36), demand new methodologies that can efficiently couple both. Multiplexing, as defined by the simultaneous evaluation of several experimental elements, can accomplish this goal (33,37). Multiplexing allows for a significant increase in the number of samples analyzed per unit of time. When high-throughput screening is paired with multiplexing, time efficiency is enhanced while cost can be considerably reduced (22,38,39). Krutzik and Nolan (22,33) describe an elegant way of multiplexing cell analysis aimed at distinguishing different cell populations based on increasing amount of antibody/stain. While this approach does decrease time, it relies on previous careful laborious calibration of the staining technique, whether it is antibody or dye-based. Moreover, the approach may be compromised with rapidly dividing cells (40) or with cells overexpressing transporter systems that interact with dyes.

### SUMMARY

The invention provides methods for tagging, uniquely identifying or genetically barcoding a cell, a population of cells, or a culture of cells, comprising: (a) providing a cell, a population of cells or a culture of cells; (b) providing at least one nucleic acid encoding a readable or detectable moiety, wherein the at least one nucleic acid is contained within a vehicle, plasmid, vector or recombinant virus, or equivalent thereof, capable of stably transfecting, transducing, infecting or implanting in the cell; and, (c) transferring, transfecting, transducing, infecting or implanting the at least one readable or detectable moiety-encoding nucleic acid into the cell or culture of cells, thereby stably transfecting, transducing, infecting or implanting in the cell the vehicle, plasmid, vector or recombinant virus, or equivalent thereof, wherein optionally the vehicle, plasmid, vector or recombinant virus, or equivalent thereof, is stably replicated by the cell during mitosis as an autonomous structure, or is incorporated within the cell's genome.

In alternative embodiments, methods of the invention further comprise culturing the cell or culture of cells and expressing the readable or detectable moiety, or culturing the cell or culture of cells under conditions in which the readable or detectable moiety is expressed. In alternative embodiments, methods of the invention further comprise isolating, counting or characterizing the tagged, uniquely identified or genetically barcoded cell or a population of cells.

In alternative embodiments, the tagged, uniquely identified or genetically barcoded cell or a population of cells is isolated, counted or characterized using a flow cytometry or a fluorescent activated cell sorting (FACS), or equivalent. In alternative embodiments, the transferring, transfecting, transducing, infecting or implanting is in vitro, ex vivo or in vivo, and optionally the cell or the population of cells is in vivo.

In alternative embodiments, methods of the invention further comprise identifying or counting the number of readable or detectable moiety expressing cells. The readable or detectable moiety can comprise a fluorescent protein, or the nucleic acid encodes a protein detectable by a fluorescent, a luminescent, a colorimetric or an equivalent detection assay. The fluorescent protein can comprise a member of the group consisting of: a green fluorescent protein (GFP), an mCherry protein, a td Tomato protein, an E2 Crimson protein, a Cerulean protein, an mBanana protein and a combination thereof, including one, two, or three or more different readable or detectable moieties and/or readable or detectable moieties at different intensities.

In alternative embodiments, at least two, three, four or five or more different readable or detectable moieties are transferred into the cell or culture of cells; or, at least two, three, four or five or more different nucleic acids encoding different or distinguishable readable or detectable moieties are stably transfected, transduced, infected or implanted in the cell.

In alternative embodiments, each of the at least two, three, four or five or more different readable or detectable moieties has a spectrum range that does not overlap, or does not significantly overlap, with any of the other readable or detectable moieties.

In alternative embodiments, clonal cell populations carrying individual readable or detectable moieties, or fluorescent proteins, are selected based on expression at different intensities, and optionally the expression level is such that the value of the mean fluorescence intensity of each population is one log scale apart to make them distinguishable.

In alternative embodiments, the at least one nucleic acid is operably linked to an inducible or a constitutively active transcriptional activator or promoter; or, if more than one readable or detectable moieties are used, each type of nucleic acid is operably linked to its own transcriptional activator or promoter (each class of readable or detectable moieties is operatively linked to a different transcriptional activator or promoter).

In alternative embodiments, the vehicle, plasmid, vector or recombinant virus capable of stably transfecting the cell is or comprises: a recombinant retrovirus or a lentiviral recombinant virus, a Vesicular Stomatitis Virus, or a Vesicular Stomatitis Virus Envelope glycoprotein vector, or a vehicle, plasmid, vector or recombinant virus capable of targeting a particular cell type or cell phenotype.

In alternative embodiments, methods of the invention further comprise use of antibody staining to count or identify a cell or a cell population or a phenotype. In alternative embodiments, the cells are mammalian cells, animal cells, or human cells, or are cells derived from cell lines or stable cell cultures.

The invention provides cells or populations of cells tagged, uniquely identified or genetically barcoded using a method of the invention.

The invention provides non-human animals or non-human mammals comprising a cell or a population of cells tagged, uniquely identified or genetically barcoded using a method of the invention.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1A illustrates a representation of a flow cytometry analysis of the mammalian cells Huh7 5.1 and HEK 293T transduced with retroviral particles expressing nucleic acids encoding the nucleic acids as indicated in the figures, thus genetically barcoding the cells with fluorescent proteins as demonstrated by the flow cytometry analysis, as discussed in detail in Example 1, below.

FIG. 1B illustrates a representation of a flow cytometry analysis of the non-adherent SupT1 T-cell line transduced with retroviral particles expressing two fluorescent proteins in different combinations to matrix of up to four unique populations: the naïve (population #1), the single positive (populations #2 and 3) and the double positive (populations #4) expressing both cyan fluorescent protein (CFP) and mCherry, as discussed in detail in Example 1, below.

FIG. 2 illustrates a representation of flow cytometry analysis of SupT1 cells analyzed seventy-two hours following simultaneous retroviral transduction with particles carrying td Tomato and particles carrying E2 Crimson: upon transduction cells were obtained that express either td Tomato, E2 Crimson or both, as illustrated in FIG. 2, left panel, and gates were set to obtain distinct clonal populations based on fluorescence channel and intensity, as shown by the circles in the left panel; and, a single intensity was chosen for E2 Crimson and two for td Tomato (dim and high), where after sorting and amplification a matrix of six distinctive populations was obtained as labeled in FIG. 2, right panel, as discussed in detail in Example 1, below.

FIG. 3 illustrates a representation of flow cytometry analysis of SupT1 clonal populations analyzed at day zero for having six distinctive populations, as labeled in the figures, and then passaged for six months in order to determine whether protein expression was stable: FIG. 3, right upper panel, shows that the selected populations remain distinguishable over at least six months; and to corroborate that freeze-thaw does not disturb signal stability, the same cells analyzed at day zero were frozen for a period of six months, thawed and re-analyzed, and the flow cytometry analysis of FIG. 3, left panel versus FIG. 3, right lower panel show that populations do not differ from the cells passaged for the same period of time, as discussed in detail in Example 1, below.

FIGS. 4A-4B illustrate a representation of flow cytometry analysis of the panel of the six populations of cells as illustrated in FIG. 2 further enhanced with an additional fluorescent marker, where td Tomato and E2 Crimson were transduced with retroviral particles carrying enhanced GFP (eGFP), the original populations (#1-6, as shown in FIG. 4A) were compared to the eGFP-expressing “enhanced” populations (#7-12, as shown in FIG. 4B), and data show that td Tomato and E2 Crimson populations have identical signatures (FIG. 4A, dot plots in left panels), however, when analyzed for eGFP, six new populations are revealed, as illustrated in the FIG. 4A-4B histograms in the right panels, as discussed in detail in Example 1, below; and, the data show that a matrix of twelve populations can be obtained by combining the original six-population panel (as illustrated in FIG. 4A) with the same panel expressing eGFP (as illustrated in FIG. 4B), as discussed in detail in Example 1, below.

FIG. 5A-5C illustrate a representation of flow cytometry analysis demonstrating the power of deconvolution in genetically barcoded cells: as illustrated in FIG. 5A, a set of eGFP negative six populations (#1-6) was mixed with three of the eGFP expressing populations #7, 9 and 11; and as illustrated in FIG. 5B, the masked populations are revealed when analyzed for eGFP (without eGFP the new panel of nine is not distinguishable from the original panel of six analyzed for td Tomato and E2 Crimson); and, as illustrated in FIG. 5C, gating of the different eGFP positive populations allows tracking back when analyzed with the original channels, as seen by juxtaposition of the initial “colored” (td tomato) populations with the green (eGFP-enhanced) populations, as illustrated in the right panel in FIG. 5C, as discussed in detail in Example 1, below.

FIGS. 6A-6B illustrate a representation of flow cytometry analysis of genetically barcoded SupT1 cells expressing HIV-1 protease variants, where a mixed population of SupT1 cells expressing combinations of fluorescent proteins (E2 Crimson and td Tomato) and PR variants were analyzed for E2 Crimson and td Tomato (dot plot, as illustrated in FIG. 6A); and following activation with Dox and Dar, all clones turn green fluorescent, as illustrated in the histograms in the lower panels, as illustrated in FIG. 6B, as discussed in detail in Example 1, below.

FIGS. 7A-7C illustrates a representation of flow cytometry analysis of genetically barcoded SupT1 cells expressing cell surface markers through a cell-based assay; FIG. 6A illustrates a mixed population of SupT1 cells expressing td Tomato at different intensities, as analyzed in a PE channel, see left dot panel; FIG. 6B illustrates flow cytometry analysis of cells stained with anti-FLAG and anti-HA antibodies and analyzed in FITC and APC channels, see right dot plot FIG. 7C, as discussed in detail in Example 1, below. D

FIGS. 8A-8D illustrate an exemplary model of genetic barcoding of the invention for multiplexing, where deconvolution can reveal masked populations: FIG. 8A illustrates a representation of flow cytometry analysis of genetically barcoded cells expressing cell surface markers; FIG. 8B schematically illustrates a FACS analysis protocol; FIG. 8C illustrates a representation of flow cytometry analysis showing a mixed population of cells expressing combinations CFP and mCherry; and, FIG. 8D illustrates histograms of the different barcoded cell populations of FIG. 8C, as discussed in detail in Example 1, below.

FIG. 9 illustrates Table 1 and Table 2, each presenting data demonstrating the multiplexing power of exemplary assays of the invention using up to three proteins with up to two intensities each: Table 1 presents data where the #Multiplex column shows the number of possible distinguishable populations in each panel, protein C and/or D freed for biological function; and, Table 2 presents data where the #Multiplex column shows the number of possible distinguishable populations in each panel, protein C and/or D freed for biological function, as discussed in detail in Example 1, below.

Like reference symbols in the various drawings indicate like elements.

### DETAILED DESCRIPTION

The invention provides cells or populations of cells, including non-human animals or non-human mammals having these cells, where the cells or populations of cells are stably tagged, uniquely identified and genetically barcoded by one or more detectable, e.g., fluorescent, proteins; and methods of making and using them.

This invention expands the analytical capabilities of flow cytometry by exploiting the power of genetic engineering to tag or barcode individual cells or populations of cells with nucleic acids or genes encoding detectable, e.g., fluorescent, proteins. In alternative embodiments, genetic engineering with Vesicular Stomatitis Virus (VSV), or retroviral (e.g., lentiviral) technology is used to express ectopic genetic information intracellularly for tagging or barcoding the cells in a stable manner. In alternative embodiments, the cells or populations of cells are mammalian, animal or human cells. Because retroviral expression can be stable for at least a six-month period, the tagging or barcoding of the invention can be used for a variety of biological applications, e.g., such as biological screens.

We have shown the applicability of fluorescent barcoded multiplexing to cell-based assays. We have genetically barcoded both adherent and non-adherent cells with different fluorescent proteins. In alternative embodiments, multiplexing power was increased by combining both the number of distinct fluorescent proteins, and the fluorescence intensity in each channel.

The fluorescent genetic barcoding of the invention gives the cell an inherited characteristic. Once cell-lines are developed, no further manipulation or staining is required, decreasing time, non-specific background associated with staining protocols, and cost. The increasing number of discovered and/or engineered fluorescent proteins with unique absorbance/emission spectra, combined with the growing number of detection devices and lasers, increases multiplexing versatility, making this invention's fluorescent genetic barcoding a powerful tool for flow cytometry-based analysis.

In alternative embodiments, the invention retains multiplexing capabilities without the need for dyes, stains, antibodies, quantum dots or bio-labels in general. In alternative embodiments, use of retroviral-based technology allows for stable expression of ectopic genetic information, i.e., the readable or detectable moieties used to practice this invention, for long periods of time because, e.g., the information carried inside the retroviral particle is integrated into active sites of transcription, which can be as part of the virus' natural life cycle (45).

In alternative embodiments, the invention uses a green fluorescent protein (GFP), an mCherry protein, a td Tomato protein, an E2 Crimson protein, a Cerulean protein or an mBanana protein, or any equivalent fluorescent or otherwise detectable protein available for biomedical applications. In alternative embodiments, the invention's use of retroviral technology allows for the engineering of cells encoding a diverse range of detectable, e.g., fluorescent, gene products, thus generating cell populations distinguishable by their fluorescence characteristics. A distinct fluorescence profile identifies one cell from its counterpart and can thus be exploited for what we refer to as “genetic barcoding.”

In alternative embodiments, the invention's use of genetic barcoding further allows the mixing of unique fluorescent cells, dramatically increasing multiplex capabilities. In alternative embodiments, the chosen detectable, e.g., fluorescent, proteins have minimal spectral overlap, particularly when practicing the multiplexing embodiment of the invention.

In alternative embodiments, to further enhance the power of multiplexing, each cell population can harbor a number of fluorescent proteins. Each fluorescent protein can also be selected on the basis of varying fluorescence intensities. Thus, in alternative embodiments, a matrix with a larger number of distinguishable populations can be obtained by combining different fluorescent proteins and intensities. Established populations of barcoded cells with fluorescent genetic markers can be used in tandem with an array of cell-based assays to address a variety of biological questions.

### Fluorescent Proteins

In alternative embodiments, one or more fluorescent proteins are used to practice the invention. For example, assays of the invention can comprise use of one or more of: a green fluorescent protein (GFP), an Cherry protein, a td Tomato protein, an E2 Crimson protein, a Cerulean protein or an mBanana protein, or any equivalent fluorescent or otherwise detectable protein.

In alternative embodiments, a green fluorescent protein (GFP) used to practice this invention comprises 238 amino acid residues, at 26.9 kDa, that exhibits a bright green fluorescence when exposed to light in the blue to ultraviolet range. Equivalent green fluorescent proteins can have a major excitation peak at a wavelength of 395 nm and a minor one at 475 nm.; an emission peak can be at 509 nm, which is in the lower green portion of the visible spectrum. The fluorescence quantum yield (QY) of GFP is 0.79. For example, in alternative embodiments, a GFP from the jellyfish Aequorea victoria or a GFP from the sea pansy Renilla reniformis is used having a single major excitation peak at 498 nm.

Equivalent fluorescent proteins that can be used to practice the invention include any red fluorescent protein, e.g., as derived from Discosoma sp. In one embodiment, another red fluorescent protein mCherry protein is used: it is a monomeric fluorescent construct with peak absorption/emission at 587 nm and 610 nm, respectively. It is resistant to photobleaching and is stable. It matures quickly, with a to 0.5 of 15 minutes, allowing it to be visualized soon after translation.

In alternative embodiments, a tdTomato used to practice this invention is an exceptionally bright red fluorescent protein. tdTomato's emission wavelength of 581 nm and brightness make it ideal for live animal imaging studies. The tdTomato fluorescent protein is equally photostable to mCherry.

Equivalent red fluorescent proteins that can be used to practice the invention include:

| Fluorescent | Excitation | Emission |
|---|---|---|
| Protein | Maximum (nm) | Maximum (nm) |
| mCherry | 587 | 610 |
| tdTomato | 554 | 581 |
| DsRed-Monomer | 557 | 592 |
| DsRed-Express2 | 554 | 591 |
| DsRed-Express | 554 | 586 |
| DsRed2 | 563 | 582 |
| AsRed2 | 576 | 592 |
| mStrawberry | 574 | 596 |

(Clontech; Takara Bio Company; Takara Holdings Inc., Kyoto, Japan).

In alternative embodiments, E2-Crimson used to practice this invention is a bright far-red fluorescent protein initially designed for in vivo applications involving sensitive cells such as primary cells and stem cells. E2-Crimson was derived from DsRed-Express2, and retains its rapid maturation (half time of 26 minutes at 37° C.), high photostability, high solubility, and low cytotoxicity (Takara Holdings Inc., Kyoto, Japan).

In alternative embodiments, other exemplary fluorescent or otherwise detectable proteins that can be used to practice the invention include:

### Blue/UV Proteins

| | | | Extinction | | | | | |
|---|---|---|---|---|---|---|---|---|
| Protein | λₑₓ | λₑₘ | coeff. | QY | Brightness | Aggregation | pKa | Source |
| TagBFP | 402 | 457 | 52000 | 0.63 | 32.8 | Monomer | 2.7 | Evrogen |
| mTagBFP2 | 399 | 454 | 50600 | 0.64 | 32.4 | Monomer | 2.7 | [48] |
| Azurite | 383 | 450 | 26200 | 0.55 | 14.4 | Monomer | 5.0 | [28] |
| EBFP2 | 383 | 448 | 32000 | 0.56 | 18 | Monomer | 5.3 | [29] |
| mKalama1 | 385 | 456 | 36000 | 0.45 | 16 | Monomer | 5.5 | [29] |
| Sirius | 355 | 424 | 15000 | 0.24 | 3.6 | Monomer | <3.0 | [37] |
| Sapphire | 399 | 511 | 29000 | 0.64 | 18.6 | Monomer | | [8] |
| T-Sapphire | 399 | 511 | 44000 | 0.6 | 26.4 | Monomer | | [12] |

### Cyan Proteins

| | | | Extinction | | | | | |
|---|---|---|---|---|---|---|---|---|
| Protein | λₑₓ | λₑₘ | coeff. | QY | Brightness | Aggregation | pKa | Source |
| ECFP | 433 | 475 | 32500 | 0.4 | 13.0 | Monomer | 4.7 | |
| Cerulean | 433 | 475 | 43000 | 0.62 | 26.7 | Monomer | 4.7 | [13] |
| SCFP3A | 433 | 474 | 30000 | 0.56 | 16.8 | Monomer | <4.5 | [39] |
| mTurquoise | 434 | 474 | 30000 | 0.84 | 25.2 | Monomer | | [36] |
| mTurquoise2 | 434 | 474 | 30000 | 0.93 | 27.9 | Monomer | 3.1 | [47] |
| monomeric | 470 | 496 | 22150 | 0.7 | 15.5 | Monomer | 7.0 | MBL |
| Midoriishi-Cyan | | | | | | | | International |
| TagCFP | 458 | 480 | 37000 | 0.57 | 21.0 | Monomer | 4.7 | Evrogen |
| mTFP1 | 462 | 492 | 64000 | 0.85 | 54.0 | Monomer | 4.3 | Allele Biotech |

### Green Proteins

| | | | Extinction | | | | | |
|---|---|---|---|---|---|---|---|---|
| Protein | λₑₓ | λₑₘ | coeff. | QY | Brightness | Aggregation | pKa | Source |
| EGFP | 488 | 507 | 56000 | 0.6 | 33.6 | Monomer | | |
| Emerald | 487 | 509 | 57500 | 0.68 | 37.3 | Monomer | | [8] |
| Superfolder GFP | 485 | 510 | 83300 | 0.65 | 54.1 | Monomer | | [38] |
| Monomeric | 492 | 505 | 55000 | 0.74 | 40.7 | Monomer | 5.8 | MBL |
| Azami Green | | | | | | | | International |
| TagGFP2 | 483 | 506 | 56500 | 0.6 | 33.9 | Monomer | 4.7 | Evrogen |
| mUKG | 483 | 499 | 60000 | 0.72 | 43.2 | Monomer | 5.2 | [26] |
| mWasabi | 493 | 509 | 70000 | 0.80 | 56.0 | Monomer | 6.0 | Allele Biotech |
| Clover | 505 | 515 | 111000 | 0.76 | 84.4 | Monomer | 6.1 | [46] |
| mNeonGreen | 506 | 517 | 116000 | 0.80 | 92.8 | Monomer | 5.7 | [49] |

### Yellow Proteins

| | | | Extinction | | | | | |
|---|---|---|---|---|---|---|---|---|
| Protein | λₑₓ | λₑₘ | coeff. | QY | Brightness | Aggregation | pKa | Source |
| EYFP | 513 | 527 | 83400 | 0.61 | 50.9 | Monomer | | |
| Citrine | 516 | 529 | 77000 | 0.76 | 58.5 | Monomer | 5.7 | [2] |
| Venus | 515 | 528 | 92200 | 0.57 | 52.5 | Monomer | 6.0 | [9] |
| SYFP2 | 515 | 527 | 101000 | 0.68 | 68.7 | Monomer | 6.0 | [39] |
| TagYFP | 508 | 524 | 64000 | 0.62 | 39.7 | Monomer | 5.5 | Evrogen |

### Orange Proteins

| | | | Extinction | | | | | |
|---|---|---|---|---|---|---|---|---|
| Protein | λₑₓ | λₑₘ | coeff. | QY | Brightness | Aggregation | pKa | Source |
| Monomeric | 548 | 559 | 51600 | 0.6 | 31.0 | Monomer | 5.0 | MBL |
| Kusabira- | | | | | | | | International |
| Orange | | | | | | | | |
| mKOκ | 551 | 563 | 105000 | 0.61 | 64.0 | Monomer | 4.2 | [26] |
| mKO2 | 551 | 565 | 63800 | 0.62 | 39.6 | Monomer | 5.5 | MBL |
| | | | | | | | | International |
| mOrange | 548 | 562 | 71000 | 0.69 | 49.0 | Monomer | 6.5 | [16] |
| mOrange2 | 549 | 565 | 58000 | 0.60 | 34.8 | Monomer | 6.5 | [33] |

### Red Proteins

| | | | Extinction | | | | | |
|---|---|---|---|---|---|---|---|---|
| Protein | λₑₓ | λₑₘ | coeff. | QY | Brightness | Aggregation | pKa | Source |
| mRaspberry | 598 | 625 | 86000 | 0.15 | 12.9 | Monomer | | [15] |
| mCherry | 587 | 610 | 72000 | 0.22 | 15.8 | Monomer | <4.5 | [16] |
| mStrawberry | 574 | 596 | 90000 | 0.29 | 26.1 | Monomer | <4.5 | [16] |
| mTangerine | 568 | 585 | 38000 | 0.3 | 11.4 | Monomer | 5.7 | [16] |
| tdTomato | 554 | 581 | 138000 | 0.69 | 95.2 | Monomer | 4.7 | [16] |
| TagRFP | 555 | 584 | 100000 | 0.48 | 49.0 | Monomer | 3.8 | Evrogen |
| TagRFP-T | 555 | 584 | 81000 | 0.41 | 33.2 | Monomer | 4.6 | [33] |
| mApple | 568 | 592 | 75000 | 0.49 | 36.7 | Monomer | 6.5 | [33] |
| mRuby | 558 | 605 | 112000 | 0.35 | 39.2 | Monomer | 4.4 | [35] |
| mRuby2 | 559 | 600 | 113000 | 0.38 | 43 | Monomer | 5.3 | [46] |

### Far-Red Proteins

| | | | Extinction | | | | | |
|---|---|---|---|---|---|---|---|---|
| Protein | λₑₓ | λₑₘ | coeff. | QY | Brightness | Aggregation | pKa | Source |
| mPlum | 590 | 649 | | 0.1 | | Monomer | | [15] |
| HcRed-Tandem | 590 | 637 | 160000 | 0.04 | 6.4 | Monomer | | |
| mKate2 | 588 | 633 | 62500 | 0.40 | 25 | Monomer | 5.4 | Evrogen |
| mNeptune | 600 | 650 | 67000 | 0.20 | 13.4 | Monomer | 5.4 | [34] |
| NirFP | 605 | 670 | 15700 | 0.06 | 0.9 | Dimer | 4.5 | Evrogen |

### Near-IR Proteins

| | | | Extinction | | | | | | |
|---|---|---|---|---|---|---|---|---|---|
| Protein | λₑₓ | λₑₘ | coeff. | QY | Brightness | Aggregation | pKa | Source | |
| TagRFP657 | 611 | 657 | 34000 | 0.10 | 3.4 | Monomer | 5.0 | [30] | |
| IFF1.4 | 684 | 708 | 102000 | 0.077 | 7.8 | Monomer | 4.6 | [31] | Bacterial |
| | | | | | | | | | phytochrome; |
| | | | | | | | | | requires |
| | | | | | | | | | biliverdin |
| | | | | | | | | | cofactor for |
| | | | | | | | | | fluorescence |
| iRFP | 690 | 713 | 105000 | 0.059 | 6.2 | Dimer | 4.0 | [43] | Bacterial |
| | | | | | | | | | phytochrome; |
| | | | | | | | | | requires |
| | | | | | | | | | biliverdin |
| | | | | | | | | | cofactor for |
| | | | | | | | | | fluorescence |

### Long Stokes Shift Proteins

| | | | Extinction | | | | | |
|---|---|---|---|---|---|---|---|---|
| Protein | λₑₓ | λₑₘ | coeff. | QY | Brightness | Aggregation | pKa | Source |
| mKeima | 440 | 620 | 14400 | 0.24 | 3.5 | Monomer | 6.5 | MBL |
| Red | | | | | | | | International |
| LSS- | 463 | 624 | 31200 | 0.08 | 2.5 | Monomer | 3.2 | [32] |
| mKate1 | | | | | | | | |
| LSS- | 460 | 605 | 26000 | 0.17 | 4.4 | Monomer | 2.7 | [32] |
| mKate2 | | | | | | | | |
| mBeRFP | 446 | 611 | 65000 | 0.27 | 17.6 | Monomer | 5.6 | [50] |

### Photoactivatible Proteins

| | | | Extinction | | | | | | |
|---|---|---|---|---|---|---|---|---|---|
| Protein | λₑₓ | λₑₘ | coeff. | QY | Brightness | Aggregation | pKa | Source | Notes |
| PA-GFP | 504 | 517 | 17400 | 0.79 | 13.7 | Monomer | | [10] | |
| PAmCherry1 | 564 | 595 | 18000 | 0.46 | 8.3 | Monomer | 6.3 | [41] | |
| PATagRFP | 562 | 595 | 66000 | 0.38 | 25.1 | Monomer | 5.3 | [40] | |

### Photoconvertible Proteins

| | | | Extinction | | | | | |
|---|---|---|---|---|---|---|---|---|
| Protein | λₑₓ | λₑₘ | coeff. | QY | Brightness | Aggregation | pKa | Source |
| Kaede (green) | 508 | 518 | 98800 | 0.88 | 86.9 | Tetramer | 5.6 | MBL |
| | | | | | | | | International |
| Kaede (red) | 572 | 580 | 60400 | 0.33 | 19.9 | Tetramer | 5.6 | MBL |
| | | | | | | | | International |
| KikGR1 | 507 | 517 | 53700 | 0.7 | 37.6 | Tetramer | 7.8 | MBL |
| (green) | | | | | | | | International |
| KikGR1 (red) | 583 | 593 | 35100 | 0.65 | 22.8 | Tetramer | 5.5 | MBL |
| | | | | | | | | International |
| PS-CFP2 | 400 | 468 | 43000 | 0.2 | 8.6 | Monomer | | Evrogen |
| PS-CFP2 | 490 | 511 | 47000 | 0.23 | 10.8 | Monomer | | Evrogen |
| mEos2 (green) | 506 | 519 | 56000 | 0.84 | 47.0 | Monomer | 5.6 | [42] |
| mEos2 (red) | 573 | 584 | 46000 | 0.66 | 30.4 | Monomer | 6.4 | [42] |
| mEos3.2 | 507 | 516 | 63400 | 0.70 | 53 | Monomer | 5.4 | [45] |
| (green) | | | | | | | | |
| mEos3.2 (red) | 572 | 580 | 32200 | 0.55 | 18 | Monomer | 5.8 | [45] |
| PSmOrange | 548 | 565 | 113300 | 0.51 | 57.8 | Monomer | 6.2 | [44] |
| PSmOrange | 634 | 662 | 32700 | 0.28 | 9.2 | Monomer | 5.6 | [44] |

### Photoswitchable Proteins

| | | | Extinction | | | | | | |
|---|---|---|---|---|---|---|---|---|---|
| Protein | λₑₓ | λₑₘ | coeff. | QY | Brightness | Aggregation | pKa | Source | |
| Dronpa | 503 | 518 | 95000 | 0.85 | 80.7 | Monomer | | MBL | From |
| | | | | | | | | International | [19]; |

Note:
Brightness is the product of extinction coefficient and quantum yield, divided by 1000.
Note:
Brightness is the product of extinction coefficient and quantum yield, divided by 1000.

### Nucleic Acid Delivery—Gene Delivery Vehicles

In alternative embodiments, the invention provides methods for tagging, uniquely identifying or genetically barcoding a cell, a population of cells, or a culture of cells by transferring, transfecting, transducing, infecting or implanting one or more nucleic acids encoding readable or detectable, e.g., fluorescent, moieties into the cells. Any protocol, method or means of transferring, transfecting, transducing, infecting or implanting nucleic acids into cells can be used to practice this invention. For example, in practicing methods of the invention, any known construct or expression vehicle, e.g., expression cassette, plasmid, vector, virus (e.g., retroviral or lentiviral expression vectors or recombinant viruses), and the like, comprising a nucleic acid encoding a readable or detectable moiety, e.g., for use as ex vivo or in vitro gene therapy vehicles, or for expression of the a readable or detectable moiety in a target cell, tissue or organ to practice the methods of this invention, e.g., for research, diagnosis, therapy, drug discovery or transplantation.

In one aspect, an expression vehicle used to practice the invention can comprise a promoter operably linked to a nucleic acid encoding a readable or detectable moiety (or functional subsequence thereof). For example, the invention provides expression cassettes comprising nucleic acid encoding a readable or detectable moiety operably linked to a transcriptional regulatory element, e.g., a promoter.

In one aspect, an expression vehicle used to practice the invention is designed to deliver a readable or detectable moiety encoding sequence, e.g., a fluorescent protein-encoding gene, or any functional portion thereof, to a tissue or cell of an individual. Expression vehicles, e.g., vectors, used to practice the invention can be non-viral or viral vectors or combinations thereof The invention can use any viral vector or viral delivery system known in the art, e.g., adenoviral vectors, adeno-associated viral (AAV) vectors, herpes viral vectors (e.g., herpes simplex virus (HSV)-based vectors), retroviral vectors, and lentiviral vectors.

In one aspect of the invention, an expression vehicle, e.g., a vector or a virus, is capable of accommodating a full-length gene or a message, e.g., a cDNA. In one aspect, the invention provides a retroviral, e.g., a lentiviral, vector capable of delivering the nucleotide sequence encoding a readable or detectable moiety in vitro, ex vivo and/or in vivo.

In one embodiment, a retroviral or a lentiviral vector used to practice this invention is a “minimal” lentiviral production system lacking one or more viral accessory (or auxiliary) gene. Exemplary lentiviral vectors for use in the invention can have enhanced safety profiles in that they are replication defective and self-inactivating (SIN) lentiviral vectors. Lentiviral vectors and production systems that can be used to practice this invention include e.g., those described in U.S. Pat. Nos. (USPNs) 6,277,633; 6,312,682; 6,312,683; 6,521,457; 6,669,936; 6,924,123; 7,056,699; and 7,198,784; any combination of these are exemplary vectors that can be employed in the practice of the invention. In an alternative embodiment, non-integrating lentiviral vectors can be employed in the practice of the invention. For example, non-integrating lentiviral vectors and production systems that can be employed in the practice of the invention include those described in U.S. Pat. No. 6,808,923.

The expression vehicle can be designed from any vehicle known in the art, e.g., a recombinant adeno-associated viral vector as described, e.g., in U.S. Pat. App. Pub. No. 20020194630, Manning, et al.; or a lentiviral gene therapy vector, e.g., as described by e.g., Dull, et al. (1998) J. Virol. 72:8463-8471; or a viral vector particle, e.g., a modified retrovirus having a modified proviral RNA genome, as described, e.g., in U.S. Pat. App. Pub. No. 20030003582; or an adeno-associated viral vector as described e.g., in U.S. Pat. No. 6,943,153, describing recombinant adeno-associated viral vectors for use in the eye; or a retroviral or a lentiviral vector as described in U.S. Pat. Nos. 7,198,950; 7,160,727; 7,122,181 (describing using a retrovirus to inhibit intraocular neovascularization in an individual having an age-related macular degeneration); or U.S. Pat. No. 6,555,107.

Any viral vector can be used to practice this invention, and the concept of using viral vectors for gene therapy is well known; see e.g., Verma and Somia (1997) Nature 389:239-242; and Coffin et al (“Retroviruses” 1997 Cold Spring Harbour Laboratory Press Eds: J M Coffin, S M Hughes, H E Varmus pp 758-763) having a detailed list of retroviruses. Any retrovirus or lentivirus belonging to the retrovirus family can be used for infecting both dividing and non-dividing cells with a readable or detectable moiety-encoding nucleic acid, see e.g., Lewis et al (1992) EMBO J. 3053-3058.

Viruses from retrovirus or lentivirus groups from “primate” and/or “non-primate” can be used; e.g., any primate lentivirus can be used, including the human immunodeficiency virus (HIV), the causative agent of human acquired immunodeficiency syndrome (AIDS), and the simian immunodeficiency virus (SIV); or a non-primate lentiviral group member, e.g., including “slow viruses” such as a visna/maedi virus (VMV), as well as the related caprine arthritis-encephalitis virus (CAEV), equine infectious anemia virus (EIAV) and/or a feline immunodeficiency virus (FIV) or a bovine immunodeficiency virus (BIV).

In alternative embodiments, retrovirus or lentiviral vectors used to practice this invention are pseudotyped lentiviral vectors. In one aspect, pseudotyping used to practice this invention incorporates in at least a part of, or substituting a part of, or replacing all of, an env gene of a viral genome with a heterologous env gene, for example an env gene from another virus. In alternative embodiments, the lentiviral vector of the invention is pseudotyped with VSV-G. In an alternative embodiment, the lentiviral vector of the invention is pseudotyped with Rabies-G.

Retrovirus or lentiviral vectors used to practice this invention may be codon optimized for enhanced safety purposes. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms. Codon optimization has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimization also overcomes the Rev/RRE requirement for export, rendering optimized sequences Rev independent. Codon optimization also reduces homologous recombination between different constructs within the vector system (for example between the regions of overlap in the gag-pol and env open reading frames). The overall effect of codon optimization is therefore a notable increase in viral titer and improved safety. The strategy for codon optimized gag-pol sequences can be used in relation to any retrovirus.

Vectors, recombinant viruses, and other expression systems used to practice this invention can comprise any nucleic acid which can infect, transfect, transiently or permanently transduce a cell. In one aspect, a vector used to practice this invention can be a naked nucleic acid, or a nucleic acid complexed with protein or lipid. In one aspect, a vector used to practice this invention comprises viral or bacterial nucleic acids and/or proteins, and/or membranes (e.g., a cell membrane, a viral lipid envelope, etc.). In one aspect, expression systems used to practice this invention comprise replicons (e.g., RNA replicons, bacteriophages) to which fragments of DNA may be attached and become replicated. In one aspect, expression systems used to practice this invention include, but are not limited to RNA, autonomous self-replicating circular or linear DNA or RNA (e.g., plasmids, viruses, and the like, see, e.g., U.S. Pat. No. 5,217,879), and include both the expression and non-expression plasm ids.

In one aspect, a recombinant microorganism or cell culture used to practice this invention can comprise “expression vector” including both (or either) extra-chromosomal circular and/or linear nucleic acid (DNA or RNA) that has been incorporated into the host chromosome(s). In one aspect, where a vector is being maintained by a host cell, the vector may either be stably replicated by the cells during mitosis as an autonomous structure, or is incorporated within the host's genome.

In one aspect, an expression system used to practice this invention can comprise any plasmid, which are commercially available, publicly available on an unrestricted basis, or can be constructed from available plasm ids in accord with published procedures. Plasmids that can be used to practice this invention are well known in the art.

In alternative aspects, a vector used to make or practice the invention can be chosen from any number of suitable vectors known to those skilled in the art, including cosmids, YACs (Yeast Artificial Chromosomes), megaYACS, BACs (Bacterial Artificial Chromosomes), PACs (P1 Artificial Chromosome), MACs (Mammalian Artificial Chromosomes), a whole chromosome, or a small whole genome. The vector also can be in the form of a plasmid, a viral particle, or a phage. Other vectors include chromosomal, non-chromosomal and synthetic DNA sequences, derivatives of SV40; bacterial plasmids, phage DNA, baculovirus, yeast plasm ids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook. Bacterial vectors which can be used include commercially available plasmids comprising genetic elements of known cloning vectors.

### Gene Delivery Methods

The readable or detectable moiety-expressing nucleic acids used to practice the methods of the invention can be delivered for ex vivo or in vivo gene therapy to deliver the payload nucleic acid. In one aspect, readable or detectable moiety-expressing nucleic acid compositions of the invention, including non-reproducing viral constructs expressing high levels of a readable or detectable protein, are delivered ex vivo or in vivo gene therapy.

The readable or detectable moiety-expressing nucleic acids used to practice the methods of the invention can be delivered to and expressed in a variety of cell types, or specific tissues or organs, or phenotypes.

Expression of the protein can be activated by administering an activator such as a drug; e.g., through action of the drug on an inducer in the expression construct.

In one embodiment, vectors used to practice this invention, e.g., to generate a readable or detectable moiety-expressing cell, are bicistronic. In one embodiment, a MND (or, myeloproliferative sarcoma virus LTR-negative control region deleted) promoter is used to drive protein expression. In one embodiment, a reporter is also used, e.g., an enhanced green florescent protein (eGFP) reporter, which can be driven off a viral internal ribosomal entry site (vIRES). In alternative embodiments, constructs are third generation self-inactivating (SIN) lentiviral vectors and incorporate several elements to ensure long-term expression of the transgene. For example, a MND promoter allows for high expression of the transgene, while the LTR allows for long-term expression after repeated passage. In alternative embodiments, the vectors also include (IFN)-β-scaffold attachment region (SAR) element; SAR elements have been shown to be important in keeping the vector transcriptionally active by inhibiting methylation and protecting the transgene from being silenced.

The invention can incorporate use of any non-viral delivery or non-viral vector systems are known in the art, e.g., including lipid mediated transfection, liposomes, immunoliposomes, lipofectin, cationic facial amphiphiles (CFAs) and combinations thereof. Other DNA or RNA delivery techniques can also be used, such as electroporation, naked DNA techniques, gold particles, gene guns, and the like.

In one aspect, expression vehicles, e.g., vectors or recombinant viruses, used to practice the invention are injected directly into a tissue or organ. In one aspect, the readable or detectable moiety-encoding nucleic acid is administered to the individual by direct injection. Thus, in one embodiment, the invention provides sterile injectable formulations comprising expression vehicles, e.g., vectors or recombinant viruses, used to practice the invention.

In alternative embodiments, it may be appropriate to administer multiple applications and employ multiple routes, e.g., directly into the tissue and (optionally) also intravenously, to ensure sufficient exposure of target cells (e.g., stem cells or other progenitor cells) to the expression construct. Multiple applications of the expression construct may also be required to achieve the desired effect.

One particular embodiment of the invention is the ex vivo modification of stem cells of any origin or any multipotent cell, pluripotent cell, progenitor cell, or cell of a particular tissue, followed by administration of the modified cells to a non-human or mammalian host, or to any vertebrate. The cells may be directly or locally administered, for example, into a target tissue. Alternatively, systemic administration is also contemplated. The stem cells may be autologous stem cells or heterologous stem cells. They may be derived from embryonic sources or from infant or adult organisms.

In alternative embodiments, one or more “suicide sequences” are also administered, either separately or in conjunction with a nucleic acid construct of this invention, e.g., incorporated within the same nucleic acid construct (such as a vector, recombinant virus, and the like. See, e.g., Marktel S, et al., Immunologic potential of donor lymphocytes expressing a suicide gene for early immune reconstitution after hematopoietic T-cell-depleted stem cell transplantation. Blood 101:1290-1298(2003). Suicide sequences used to practice this invention can be of known type, e.g., sequences to induce apoptosis or otherwise cause cell death, e.g., in one aspect, to induce apoptosis or otherwise cause cell death upon administration of an exogenous trigger compound or exposure to another type of trigger, including but not limited to light or other electromagnetic radiation exposure.

In alternative embodiments, a readable or detectable moiety-encoding nucleic acid-comprising expression construct or vehicle of the invention is formulated at an effective amount of ranging from about 0.05 to 500 ug/kg, or 0.5 to 50 ug/kg body weight, and can be administered in a single dose or in divided doses. In one aspect, a readable or detectable moiety-encoding nucleic acid-comprising expression construct or vehicle of the invention is formulated at a titer of about at least 10¹⁰, 10¹¹, 10¹², 10¹³, 10¹⁴, 10¹⁵, 10¹⁶, or 10¹⁷ physical particles per milliliter. In one aspect, the PIM-1 encoding nucleic acid is administered in about 10, 20, 30, 40, 50, 60, 70, 80, 90, 100, 110, 120, 130, 140 or 150 or more microliter (μl) injections. Doses and dosage regimens can be determined by conventional range-finding techniques known to those of ordinary skill in the art. For example, in alternative embodiments, about 10⁶, 10⁷, 10⁸, 10⁹, 10¹⁰, 10¹¹, 10¹², 10¹³, 10¹⁴, 10¹⁵, 10¹⁶ or 10¹⁷ viral (e.g., lentiviral) particles are delivered to the individual (e.g., a non-human subject, e.g., a mammalian research animal, e.g., a mouse) in one or multiple doses.

In other embodiments, a single administration (e.g., a single dose) comprises from about 0.1 μl to 1.0 μl, 10 μl or to about 100 μl. Alternatively, dosage ranges from about 0.5 ng or 1.0 ng to about 10 μg, 100 μg to 1000 μg of readable or detectable moiety-expressing nucleic acid is administered; either the amount in an expression construct, or as in one embodiment, naked DNA is injected. Any necessary variations in dosages and routes of administration can be determined by the ordinarily skilled artisan using routine techniques known in the art.

In one embodiment, a readable or detectable moiety-expressing nucleic acid is delivered in vivo directly to a target tissue or organ using a viral stock in the form of an injectable preparation containing pharmaceutically acceptable carrier such as saline. The final titer of the vector in the injectable preparation can be in the range of between about 10⁸ to 10¹⁴, or between about 10¹⁰ to 10¹², viral particles; these ranges can be effective for gene transfer.

In one aspect, readable or detectable moiety-expressing nucleic acids (e.g., vector, transgene) constructs are delivered to a target tissue or organ by direct injection, e.g., using a standard percutaneous hypodermic needle, or using catheter based methods under fluoroscopic guidance. Alternatively, readable or detectable moiety-expressing nucleic acids (e.g., vector, transgene) constructs are delivered to organs and tissues using a delivery-facilitating moiety, e.g., lipid-mediated gene transfer.

The direct injection or other localized delivery techniques can use an amount of polynucleotide or other vector that is sufficient for the readable or detectable moiety-expressing nucleic acids (e.g., vector, transgene) to be expressed to a degree which allows for sufficiently efficacy; e.g., the amount of the readable or detectable moiety-expressing nucleic acid (e.g., vector, transgene) injected in a particular tissue or organ can be in the range of between about 10⁸ to 10¹⁴, or between about 10¹⁰ to 10¹², viral particles. The injection can be made deeply into the tissue in a single injection, or be spread throughout the tissue with multiple injections. Where there is a particular area of injury or a defined area otherwise needing treatment, direct injection into that specific area may be desirable. Use of balloon catheters or other vasculature-blocking techniques to retain the polynucleotide or other vector within the area of desired treatment for a length of time can also be used.

### Multiplexed Cell Sorting

In alternative embodiments, the tagged, uniquely identified or genetically barcoded cells or a population of cells are isolated, counted or characterized using a flow cytometry or a fluorescent activated cell sorting (FACS), or equivalent. Fluorescence activated cell sorting (FACS), also called flow cytometry, can be used to sort individual cells on the basis of optical properties, including fluorescence.

In alternative embodiments, individual genetically barcoded clones with different fluorescent characteristics can be mixed together without losing their distinct characteristics. In alternative embodiments, individual clones are engineered to express more than one fluorescent protein, wherein their individual spectra do not overlap. When the fluorescence characteristics are separated, e.g., by cell sorting, one can then obtain any of their combinations together to further increase the number of populations with unique signatures.

In alternative embodiments, distinct genetically barcoded cells can be further expanded for multiplexing by exploiting the level of protein intensity. This can be achieved if the clonal populations carrying individual proteins are selected based on expression at different intensities. The expression level can be such that the value of the mean fluorescence intensity of each population is far enough apart, typically one log scale, to make them distinguishable.

When appropriate or desired, e.g., for evaluating a biological function or a phenotype evolution, genetic barcoding can be used in tandem with classical methods of antibody staining.

The invention will be further described with reference to the following examples; however, it is to be understood that the invention is not limited to such examples.

### EXAMPLES

### Example 1

### Exemplary Assays of the Invention

This invention expands the analytical capabilities of flow cytometry by exploiting the power of genetic engineering to barcode individual cells with genes encoding detectable, e.g., fluorescent, proteins. We have genetically barcoded cells with different fluorescent proteins, tested their stability across multiple generations and obtained distinct clonal populations based on differential fluorescent intensities. Moreover, to demonstrate biological applications, we established genetically barcoded cells that are adapted to existing cell-based assays, as described e.g. in Stolp, et al., “A Novel Two-Tag System for Monitoring Transport and Cleavage through the Classical Secretory Pathway—Adaptation to HIV Envelope Processing”; Plos One 2013; 8:e68835; and Hilton, et al., “An assay to monitor HIV-1 protease activity for the identification of novel inhibitors in T-cells”; Plos One 2010; 5:e10940 (46,47). In alternative embodiments, genetically barcoded cells are coupled to cell-based assays to enhance high throughput capabilities by reducing the number of screens needed.

### Materials and Methods

Cell Maintenance: HEK293T and Phoenix GP cell lines were maintained at 37° C. and 5% CO₂ in Dulbecco's Modified Eagle Medium (DMEM) (Cellgro) supplemented with 10% fetal calf serum, penicillin-streptomycin, and 2 mM L-glutamine. SupT1 cells were maintained at 37° C. and 5% CO₂ in RPMI (Cellgro) supplemented with 10% fetal calf serum, penicillin-streptomycin, and 2 mM L-glutamine. Cells were routinely screened for mycoplasma contamination. Phoenix GPs were provided by Gary Nolan from Stanford University.

Plasmid Construction: The construct pBMN-i-td Tomato was created by digesting a previously constructed plasmid pBMN-i-eGFP. TD Tomato (kindly provided by Roger Tsien at UCSD, was PCR amplified using the forward primer TATAACATGTCAATTGCCACCATGGTGAGCAAGGGCGAGGAG (SEQ ID NO:1), which contains a Pcil site, and the reverse primer ATGGACCAGCTGTACAAGTAGGTCGACTATA (SEQ ID NO:2), which contains a Sall site. The amplicon was digested with Pcil and Sall and used to ligate into pBMN-i-eGFP digested with NcoI and SalI, which removes eGFP. pBMN-i-E2 Crimson was constructed similarly. The forward primer used to amplify E2 Crimson (obtained from Clontech) was TATACCACCATGGATAGCACTGAGAACGTC (SEQ ID NO:3), containing an NcoI site and the reverse primer CGCCACCACCTGTTCCAGTAGTCTAGAGTCGACTATA (SEQ ID NO:4), which contains a Sall site. Both pBMN-i-eGFP and E2 Crimson products were digested with NcoI and SalI for ligation.

Generation of Infectious Viral Particles: For production of MLV, the Phoenix GP cell-line at 60-70% confluence was transfected with 3 μg of transfer vector (pBMN-i-E2 Crimson, pBMN-i-td Tomato, pBMN-i-eGFP, pBMN-i-eCFP) and 3 μg of Vesicular Stomatitis Virus Envelope glycoprotein vector (pCl-VSVg). DMEM media was replaced 24 hours post-transfection and viral supernatant was collected 48 hours and at 72 hours after transfection. All viral stocks were filtered with 0.45 micron PTFE filters (Pall Corporation) and frozen at −80° C. in 2 mL aliquots.

Transductions: Huh 7.5.1 and HEK 293-T cells grown in DMEM at 250,000 cells/well in a six well plate were prepared for transduction. 24 hours after plating, cells were treated with 5 μg/mL Polybrene (Hexadimethrene Bromide, Sigma) and transduced with viral stocks by hanging bucket rotors centrifuge (Becton Dickinson) at 1500 g, for 120 minutes at 32° C. 24 hours post transduction media was changed. 5×10⁶ SupT1 cells/well in a six well plate grown in RPMI supplemented were treated with 5 μg/mL Polybrene (Hexadimethrene Bromide, Sigma) and infected with viral stocks by centrifugation in a hanging bucket rotors centrifuge (Becton Dickinson) at 1500 g, for 120 minutes at 32° C. Cells were then analyzed for expression 72 hours post-infection.

Staining for analysis: Cells were pelleted and incubated with mouse anti-FLAG (Sigma Aldrich, St. Louis, Mo.) and rabbit anti-HA (Cell Signaling, Beverly, Mass.) at 1:400 dilution for 20 minutes and then washed with PBS. Cells were then incubated with anti-mouse ALEXA FLUOR 488™ and anti-rabbit ALEXA FLUOR 647™ (Cell Signaling, Beverley, Mass.) antibodies at 1:200 dilutions for 20 minutes and washed with PBS.

Flow Cytometry and Sorting: Cell samples were washed twice with 1× PBS prior to loading into the instrument. The Flow Cytometry Core Facility at San Diego State University performed analysis of cells on BD FACS Aria at 405 nm, 488 rim, and 633 nm lasers, as well as the BD FACS Canto using the 488 blue laser, and the 633 red laser. Analysis of APC, PE, and FITC channels was performed with band pass filters of 660/20, 585/42, and 530/30, respectively. Data was collected FACS DIVA 6.1.1™ software (Becton Dickinson, Franklin Lakes, N.J.), and analyzed on FLOWJO™ (Tree Star, Inc., Ashland, Oreg.).

### Results

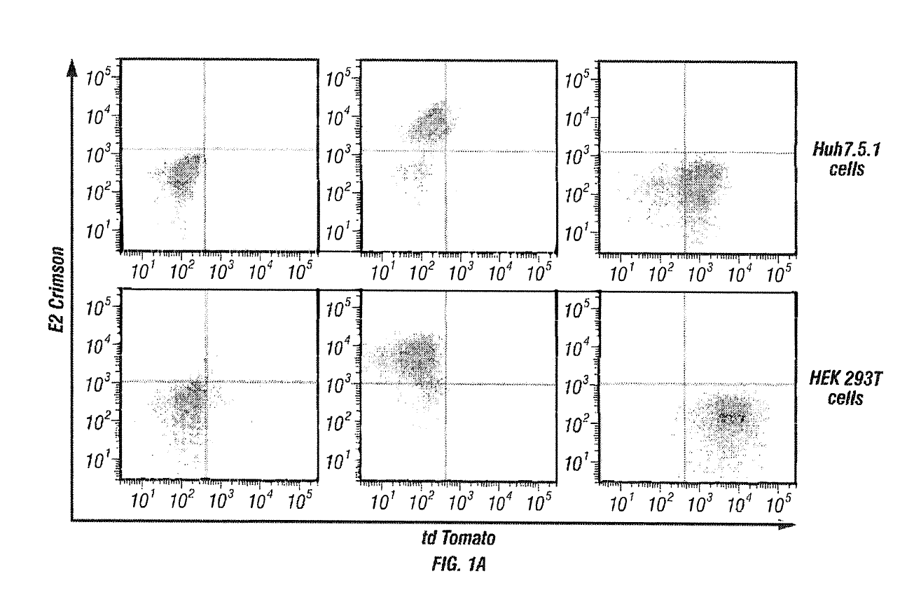

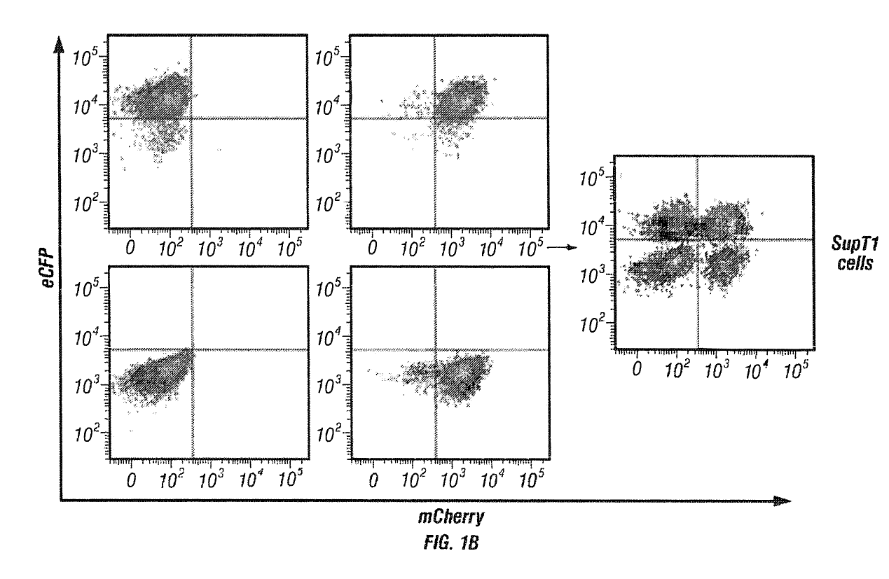

Genetically Barcoded Mammalian Cells Can Distinguish Different Populations: Individual genetically bar-coded cells must be independently obtained prior to being able to discriminate single cell populations within a mixture of cells. Expression of fluorescent proteins in mammalian cells has been extensively performed in the past (2,48-51), but not in the context of ‘genetic barcoding’. Here, we have genetically engineered cells with different fluorescent proteins with the ultimate goal of achieving genetic multiplexing capabilities. We initially selected the Huh 7.5.1 hepatocytic cell line and the commonly used human embryonic kidney (HEK) 293T cell line as examples of adherent cells to demonstrate the versatility of genetic bar-coding. Cells were transduced with retroviral particles carrying an individual fluorescent protein chosen from a variety of fluorescent proteins such as mCherry, td Tomato, and E2 Crimson. Following a process of transduction and amplification, individual cells were collected in single wells of 96-well plates using fluorescent activated cell sorting (FACS). A series of Huh 7.5.1 and HEK 293T cell clones expressing a single fluorescent protein were obtained. Mammalian cells genetically barcoded with fluorescent proteins, as shown in FIG. 1A, can be identified through flow cytometry. Visualization is only possible if cell populations are uniform and have fluorescent intensities distinguishable from the non-barcoded naïve cells, in the same channel.

Barcoding Mammalian Cells Allows for Multiplex Analysis: The generation of individual genetically barcoded clones with different fluorescent characteristics allows us to mix them together without losing their distinct characteristics. Moreover, individual clones can be engineered to express more than one fluorescent protein, provided their spectrum does not overlap. When the fluorescence characteristics are separated, one can then obtain any of their combinations together to further increase the number of populations with unique signatures. Here we have genetically barcoded a third cell type to establish the principle for higher throughput applications that avoid re-suspension of adherent cells. We chose the non-adherent SupT1 T-cell line. FIG. 1B shows that with two fluorescent proteins one can obtain a matrix of up to four unique populations; the naïve (population #1), the single positive (populations #2 and 3) and the double positive (populations #4) expressing both cyan fluorescent protein (CFP) and mCherry.

Genetically barcoded cells can be further differentiated based on fluorescence intensity: A panel of distinct genetically barcoded cells can be further expanded for multiplexing by exploiting the level of protein intensity. This can be achieved if the clonal populations carrying individual proteins are selected based on expression at different intensities. The expression level needs to be such that the value of the mean fluorescence intensity of each population is far enough apart, typically one log scale, to make them distinguishable. SupT1 cells were analyzed seventy-two hours following simultaneous retroviral transduction with particles carrying td Tomato and particles carrying E2 Crimson. As expected, upon transduction cells were obtained that express either td Tomato, E2 Crimson or both (FIG. 2, left panel). Gates were then set to obtain distinct clonal populations based on fluorescence channel and intensity, as shown in FIG. 2, left panel. As proof of principle, a single intensity was chosen for E2 Crimson and two for td Tomato (dim and high). After sorting and amplification, a matrix of six distinctive populations was obtained, including E2 Crimson (population #4), td Tomato dim and high (populations #2 and 3) and two populations expressing E2 Crimson in conjunction with mid and high td Tomato (populations #5 and #6 respectively, FIG. 2, right panel).

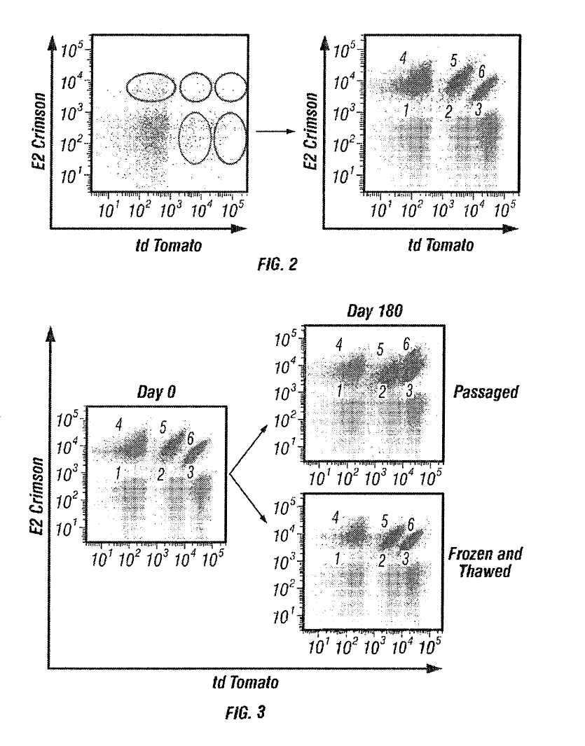

Genetically Barcoded Cells Are Stable for Long Periods of Time: The functionality of genetically barcoded cells can be further exploited for biological applications as long as the expression levels and fluorescent characteristics remain stable. To ensure the stability of genetically barcoded cells and the reproducibility of the instrumentation to identify and track populations over long periods of time, we performed a time course experiment. SupT1 clonal populations were analyzed at day zero, and passaged for six months in order to determine whether protein expression is stable. FIG. 3 (right upper panel) shows that the selected populations remain distinguishable over at least six months, proving the stability of barcoded cells and ability for performing multiplex analysis for long term usage. While population #5 (dim td Tomato/E2 Crimson) and #6 (bright td Tomato/E2 Crimson) start to merge, they are still distinguishable; with PE-A mean fluorescence intensity (MFI) values are ^{˜}3,400 and 23,000, respectively. The rest of the cell populations drifted minimally and populations #2 and #3 remained identical after six months. In order to corroborate that freeze-thaw does not disturb signal stability, the same cells analyzed at day zero were frozen for a period of six months, thawed and re-analyzed. Results show that populations do not differ from the cells passaged for the same period of time (FIG. 3, left panel versus right lower panel). Genetically bar-coded cells retain their unique fluorescent profiles and can be used in assays immediately upon thawing.

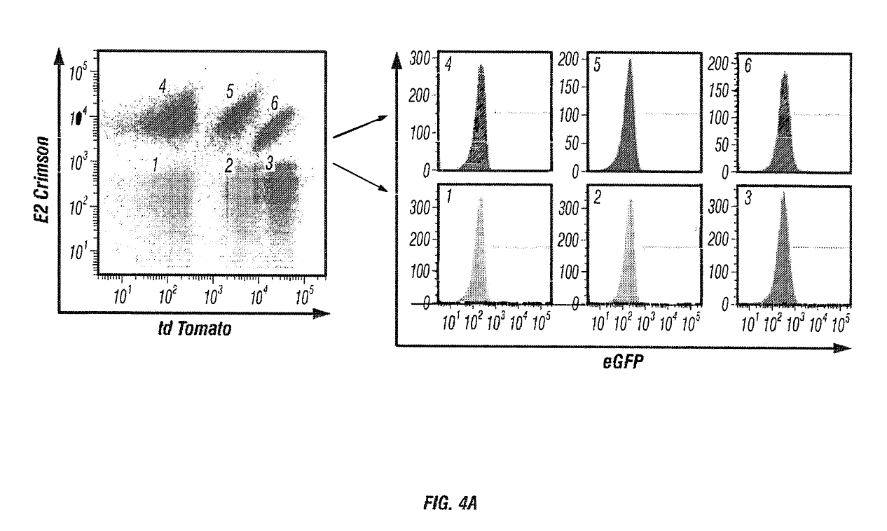

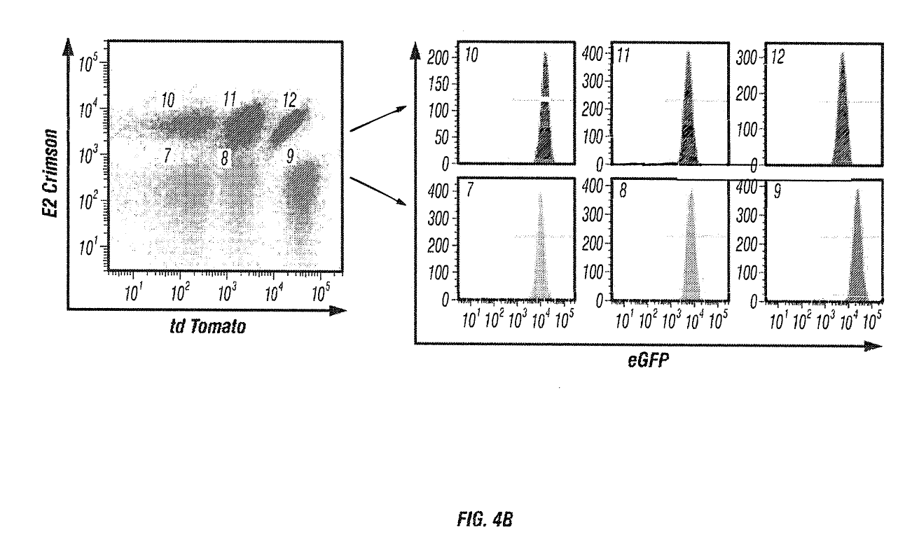

Deconvolution Allows to Further Increase Multiplexing and to Pinpoint Masked Populations. Multiplexing, as assessed in FIG. 2 with a panel of six populations, can be further enhanced with an additional fluorescent marker. The six populations obtained with a combination of td Tomato and E2 Crimson were transduced with retroviral particles carrying enhanced GFP (eGFP). Clonal populations were obtained as described previously and analyzed for E2 Crimson and td Tomato. The original populations (#1-6) were compared to the eGFP-expressing populations (#7-12). When analyzing by td Tomato and E2 Crimson the populations have identical signatures (FIG. 4, dot plots in left panels), however, when analyzed for eGFP, six new populations are revealed (FIG. 4, histograms in right panels).

The addition of eGFP allowed expansion of the matrix from a six-population (E2 Crimson and td Tomato) to a twelve-population panel (E2 Crimson, td Tomato and eGFP) in the APC, PE and FITC channels respectively. A matrix of twelve populations could thus be theoretically obtained by combining the original six-population panel (FIG. 4A) with the same panel expressing eGFP (FIG. 4B).

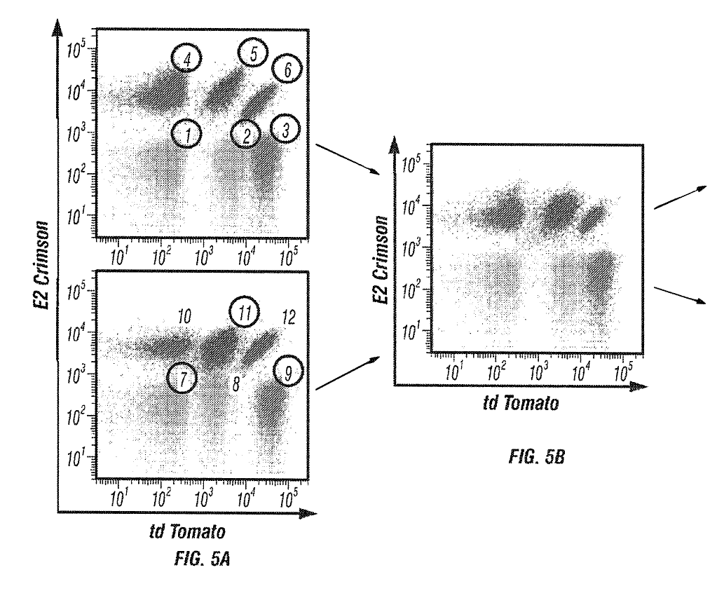

In order to demonstrate the power of deconvolution in genetically barcoded cells, we also mixed the set of eGFP negative six populations (#1-6) with three of the eGFP expressing populations (#7, 9 and 11, FIG. 5A). While the new panel of nine is not distinguishable from the original panel of six analyzed for td Tomato and E2 Crimson (FIG. 5B), the masked populations are revealed when analyzed for eGFP (FIG. 5C). Gating of the eGFP positive populations allows tracking them back and reveals them when analyzed with the original channels, as seen by juxtaposition of the initial colored populations with the green populations (right panel in FIG. 5C).

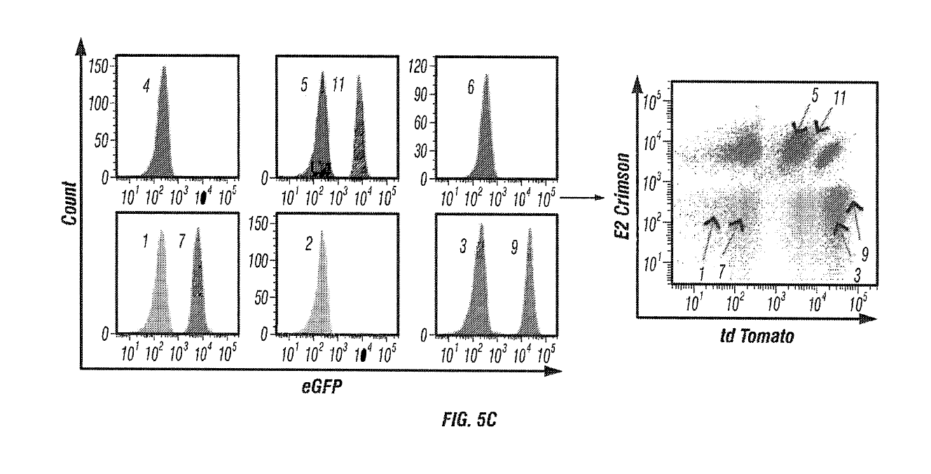

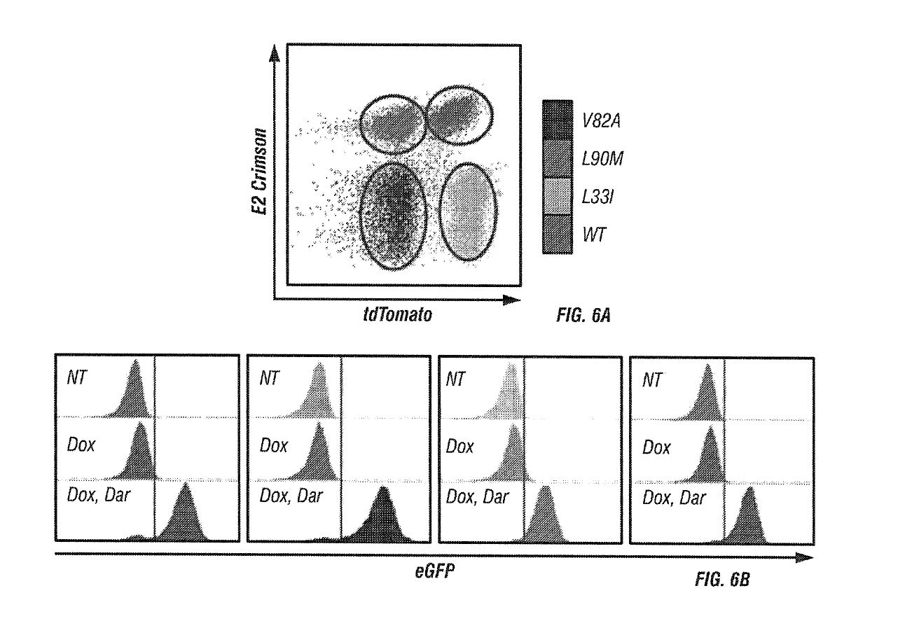

Multiplexing through Genetic Barcoding for Biological Applications—Adaptation to Cell Based Assays: As described in FIGS. 4 and 5, a panel of distinct populations based on two fluorescent proteins was further expanded with the use of a third fluorescent protein. While the third fluorescent marker was used purely as an additional genetic marker, it can be exploited as part of biological application such as cell signaling, phenotypic outcomes, or a cell-based assay. For these types of applications eGFP expression is intended to be a function of biological activity. As proof of principle we have utilized a panel of four SupT1 populations including naive, E2 Crimson, td Tomato, and E2 Crimson/td Tomato barcoded cells. Each of the populations within the panel was used to express one of four different HIV-1 protease variants in an inducible manner. In the assay, as described elsewhere (47), the protease variants can induce eGFP expression if inhibited. FIG. 6 shows the panel of genetically barcoded cells, each expressing a distinct HIV-1 protease variant. When untreated or when treated with doxycycline, neither of the populations is fluorescent as they do not express eGFP. When inhibited with a known protease inhibitor such as darunavir (e.g., PREZISTA™), all populations express eGFP, as observed upon deconvolution. Such an experiment allows simultaneous monitoring of the activity of four selected HIV-1 protease mutants using eGFP as a biosensor for protease inhibition. Deconvolution can thus reveal which population or protease variant was affected by that specific treatment, even though in this example all populations respond to inhibitor.

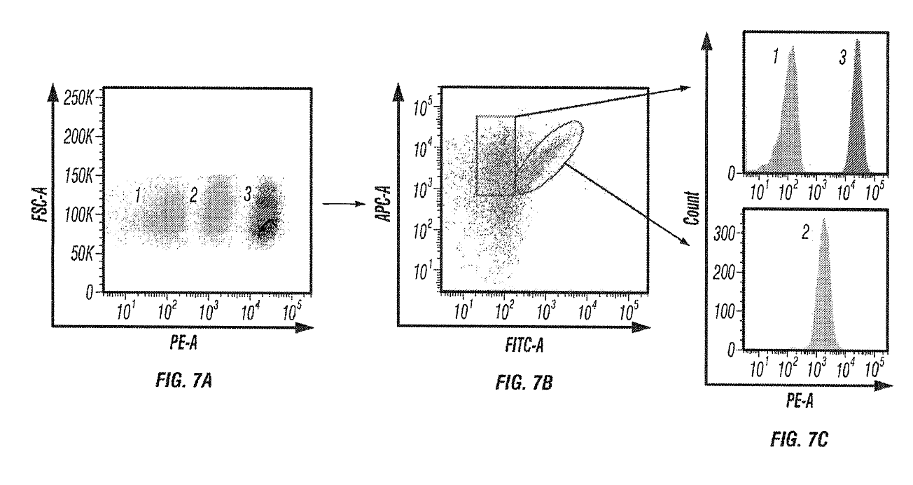

When appropriate for evaluating biological function, genetic barcoding can be used in tandem with classical methods of antibody staining. In yet another example, enhanced genetic barcoding was utilized for a cell-based assay that relies on the expression of one or two tags on the cell surface (HA or HA and FLAG, respectively). Td Tomato barcoded cells (FIG. 2, populations #2 and 3) were chosen to independently express viral proteins known to be cleaved by host enzymes (46). When cleaved, only expression of HA can be detected on the cell surface through APC-coupled anti-HA antibody (populations #1 and 3). However, if un-cleaved both HA and FLAG (FITC-coupled anti-FLAG antibody) will be detected (pop #2). The genetic elements of the assay with three different viral proteins were introduced into naïve, and dim and high td Tomato-expressing cells. The three populations carrying the assay are shown in the PE channel (FIG. 7A). When the mixed sample was analyzed in the FITC and APC channels, a combination of APC only-and APC and FITC-positive populations was observed (FIG. 7A). To further demonstrate deconvolution through genetic barcoding in the context of a cell-based assay, these cells were gated based on HA-single positive or HA and FITC-double positive staining, and analyzed in the PE channel (FIG. 7B). Distinct phenotypes could thus be observed in different subsets, which could be pinpointed and tracked through deconvolution.

### Discussion

Fluorescent protein-based genetic barcoding provides a cell with an inherited characteristic that can distinguish it from its counterparts. While labor intensive at first, it becomes valuable once cell-lines are developed, as from then on, no further manipulation or staining is required, dramatically decreasing time, non-specific background associated with staining protocols, and, of course cost. Here we have utilized the power of retroviral technology to genetically engineer a number of cell types, both adherent and non-adherent, including HEK 293T and Huh7.5.1 cells, as well as SupT1 cells, respectively. We have utilized CFP in combination with mCherry or E2 Crimson in combination with td Tomato to obtain at least a minimum of four independent populations which include naïve, two single positive and a double positive population. Expression of fluorescent proteins through retroviral technology, which exploits the ability of viral particles to insert their genome into the genome of the host, has proven to be stable for long periods of time, at least for up to six months. Stable expression further allowed us to choose a larger number of genetically barcoded cells not only based on the type of fluorescent protein but on fluorescence intensity as well.

Variations in fluorescence intensities are expected in genetic barcoding due to the inherited characteristic of retroviral technology. Effects of insertional preferences into the host genome will lead to differential expression of the fluorescent protein marker. The nature of the insertion can be exploited to increase the multiplex power of genetic barcoding through retroviral technology. Combining fluorescent proteins with distinguishable physical parameters (absorbance/emission spectra), each at different intensities, allows a matrix of a large number of distinct populations to be obtained.

Here we have shown matrixes of four, six, nine and twelve-populations, which included two six-population panels of E2 Crimson and td Tomato, with or without eGFP. As proof of principle in our example we utilized only one intensity for E2 Crimson and eGFP, and two for td Tomato. Theoretically, one could achieve a panel of up to 27 populations with three fluorescent proteins, each at two intensities. As mentioned, at least with SupT1 cells, it might be possible to expand the matrix to 27 populations considering the uniformity and the range of MFI values of the individual populations. Table 1 shows the multiplexing capabilities of combining up to three fluorescent proteins (proteins A, B and C) with up to two intensities each (low and high). We have shown panels of 4, 6 and 12 possible combinations (indicated by a star in Table 1), within the many possible combinations. The third and/or fourth color (proteins C and/or D) could be used as a marker of cell response or biological function. A panel of 81 distinct populations is theoretically possible with four colors, each at two intensities (not shown in Table 1).

While we have obtained two clear distinct intensities for td Tomato (dim and high in FIG. 2), up to three distinct intensities should be easily achievable for each protein. The theoretical multiplexing capabilities with proteins with up to three intensities each (low, medium and high) are depicted in Table 2. A major advantage of utilizing fewer proteins at more intensities rather than more proteins at fewer intensities while maintaining the same multiplexing power, is the freedom of additional channels that may be needed for biological outcomes.

The single positive td Tomato populations of low and high intensities (FIG. 2) exhibit a PE-A MFI value of ^{˜}5,250 and 31,500 respectively. However, the E2 Crimson positive cells display a maximum APC-A MFI value of ^{˜}11,850, which leads us to believe that much higher expressing populations could be obtained. While the E2 Crimson intensity of population #5 (FIG. 3) decreased over time, with APC-A MFI values decreasing from ^{˜}11,800 to ^{˜}4,200, the reduction did not hinder the ability to distinguish the populations when analyzed simultaneously. We foresee that in many real life experiments a panel of two, three or four distinct genetically barcoded populations will answer all the requirements and fulfill the advantages of multiplexing. For example, in a screen aimed at finding antivirals against Dengue virus, a panel of four would suffice to perform a screen against all the main Dengue virus serotypes. This is in considerable contrast to performing four independent screens against each of the individual serotypes. When needed, six, twelve or even a larger number of populations can be obtained.

A growing number of biological applications in clinical and research settings, in parallel to the growing technological capabilities of the available instrumentation (25,34-36), demand new methodologies that can efficiently couple both, such as the multiplexing assays of this invention. By providing multiplexing through genetic barcoding, this invention answers this demand.

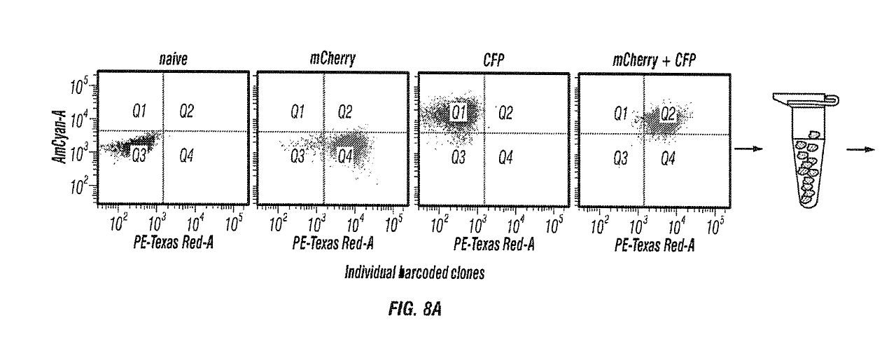

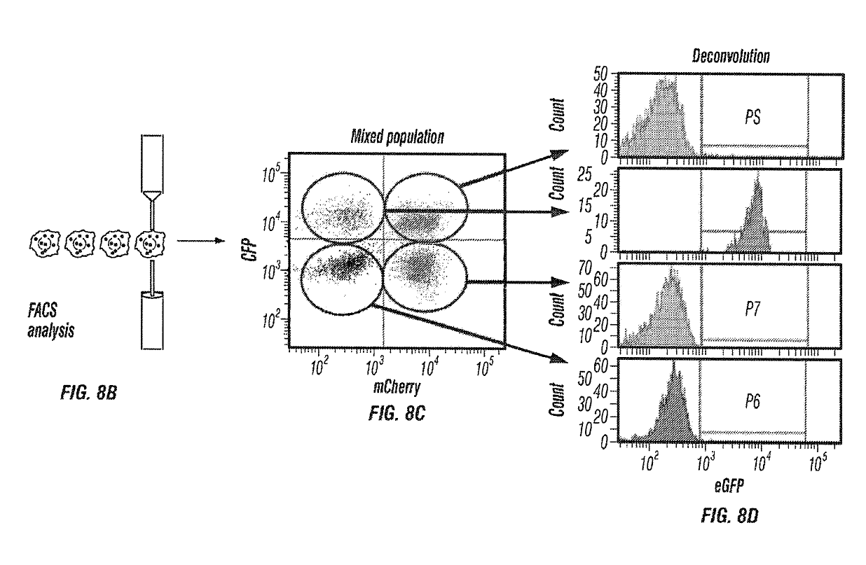

The genetic barcoding of this invention can be easily adapted to a broad range of biological applications. While in many of these applications, such as the study of signaling cascades or detection of specific endogenous markers, it is difficult if not impossible to avoid staining protocols, coupling it to genetic barcoding can still dramatically decrease cost and time. Cell-based assays that rely by themselves on fluorescent genetic markers can be coupled to genetically barcoded cells to increase the power of multiplexing, as shown in our example with HIV-1 protease variants (FIG. 6). This is also true when coupled to assays that rely on staining protocols, as demonstrated by the cell-surface marker assay (FIG. 7). The power of the multiplexed genetic barcoding of this invention is of great value for high throughput screening (HTS) applications, as the number of screens required proportionally decreases with the number of genetic barcoded populations that compose the mixed sample under analysis. The two exemplary high throughput assays of the invention described here are just two exemplary assays of a broad genus of assays provided by the invention, and all demonstrate the value that multiplexing through genetic barcoding has to accelerate drug screening and biological assays with flow cytometry-based read outs. FIG. 8 depicts the model of genetic barcoding for multiplexing, where deconvolution can reveal masked populations.

In alternative embodiments, high throughput repeatability of an assay may rely on the instrument chosen to be used and its capacities; and reproducibility of the experiment can rely on the operator. Genetic barcoding of this invention decreases the variability introduced by the human factor. The inherited properties of the genetically fluorescent barcoded cells of this invention make their detection and analysis quick, simplified and straightforward. An individual genetic background represented by a specific fluorescent protein, distinguishes the barcoded cell from its counterparts.

In alternative embodiments, increasing genetic barcoding-based multiplexing capabilities can be accomplished by meeting criteria as described e.g., by Shaner et al. (52). The growing number of existing fluorescent proteins and derivatives with distinct absorbance/emission spectra, combined with the growing number of affordable detection devices and lasers, increases the versatility of multiplexing of this invention, making fluorescent genetic barcoding a powerful tool for flow cytometry-based analysis.

In alternative embodiments, multiplexed genetic barcoding of the invention is used with assays or applications comprising flow cytometry, imaging-coupled flow-cytometry and microscopy.

### FIGURE LEGENDS

FIG. 1. Genetic barcoding of mammalian cells analyzed by flow cytometry. A. Genetic barcoded adherent cells. Huh 7.5.1 cells (upper panels) and HEK 293T cells (lower panels) clonally sorted for td Tomato or E2 Crimson. B. Multiplexed analysis of genetic barcoded non-adherent cells. Naive, CFP, mCherry, and CFP/mCherry positive SupT1 clonal populations (left panel) were mixed and analyzed by flow cytometry (right panel).

FIG. 2. Expansion of unique barcoded SupT1 populations based on fluorescence intensity. Cells were analyzed by flow cytometry three days post transduction with td Tomato and E2 Crimson viral particles. Gates were chosen (circles in left panel) to collect individual cells in order to obtain five distinct clonal populations in addition to naïve cells (right panel).

FIG. 3. Analysis of SupT1 clonal populations over a period of six months. Cells were analyzed at day 0 for E2 Crimson and td Tomato (left panel), and reanalyzed after passaging for six months (right upper panel) or frozen at −70° C. for the same period of time and thawed (right lower panel).

FIG. 4. Deconvolution of the six-population panels analyzed in the FITC channel. A. The original six populations analyzed for E2 Crimson and td Tomato (dot plot in left panel) were analyzed for eGFP (histograms in right panels). B. As in A but transduced with eGFP viral particles.

FIG. 5. Deconvolution further increases the power of multiplexing and a panel of populations can actually mask a higher number of distinct populations. A. The two six-population panels (eGFP negative: top panel, and eGFP positive: lower panel) are indistinguishable when analyzed for E2 Crimson and td Tomato. B. A mixture of nine selected populations (circled numbers in A) were analyzed for E2 Crimson and td Tomato. C. When analyzed in the FITC channel for eGFP expression, the masked populations (pop #7, 9 and 11) are revealed. Gating the eGFP positive populations allows observation of all nine populations in the same dot plot panel through artificial color juxtaposition (right panel). (Overlaying histograms results in the loss of the scale).

FIG. 6. Genetically barcoded SupT1 cells expressing HIV-1 protease variants. A mixed population of SupT1 cells expressing combinations of fluorescent proteins (E2 Crimson and td Tomato) and PR variants were analyzed for E2 Crimson and td Tomato (dot plot). Following activation with Dox and Dar, all clones turn green fluorescent (histograms in lower panels). Dox: doxycycline, Dar: darunavir.

FIG. 7. Genetically barcoded SupT1 cells expressing cell surface markers through a cell-based assay. A. A mixed population of SupT1 cells expressing td Tomato at different intensities were analyzed in the PE channel (left dot panel). B. Following staining with anti-FLAG and anti-HA antibodies, cells were analyzed in the FITC and APC channels (right dot plot). The HA-single positive and the HA and FITC-double positive populations were gated and analyzed in the PE channel to determine their barcoded identity.

FIG. 8. Depiction of genetic barcoding for multiplexing, where masked populations can be revealed through deconvolution.

### TABLES

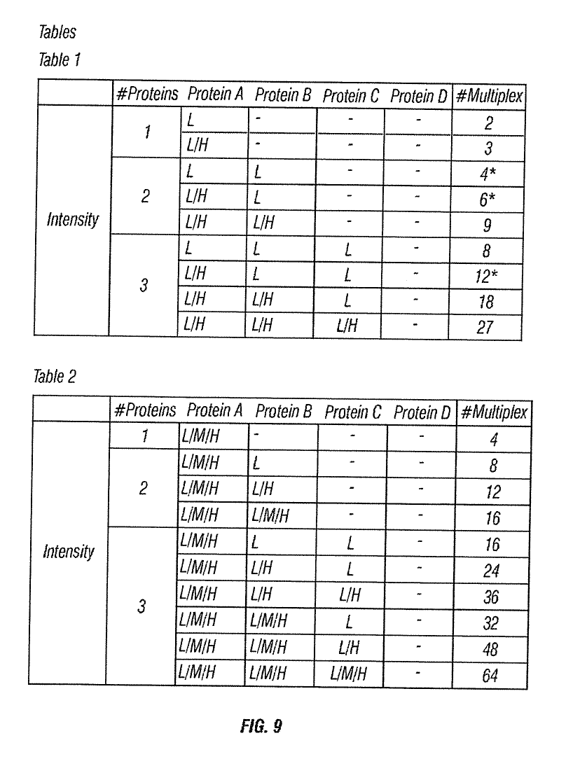

Table 1 (illustrated as FIG. 9) Multiplexing power of assays of the invention using up to three proteins with up to two intensities each. The #Multiplex column shows the number of possible distinguishable populations in each panel. Protein C and/or D freed for biological function. A panel of 81 is theoretically possible with four colors, each at two intensities (not shown). L: Low, H: High. *: Examples shown in the manuscript.

Table 2 (illustrated as FIG. 9) Multiplexing power of assays of the invention using up to three proteins with up to three intensities each. The #Multiplex column shows the number of possible distinguishable populations in each panel. Protein C and/or D freed for biological function. L: Low, M: Medium, H: High.

### REFERENCES FOR EXAMPLE 1

1. Prasher D C, Eckenrode V K, Ward W W, Prendergast F G, Cormier M J. Primary structure of the Aequorea victoria green-fluorescent protein. Gene 1992; 111:229-233.

2. Chalfie M, Tu Y, Euskirchen G, Ward W W, Prasher D C. Green fluorescent protein as a marker for gene expression. Science 1994; 263:802-805.

3. Wolkowicz R, Nolan G P, Curran M A. Lentiviral vectors for the delivery of DNA into mammalian cells. Methods Mol. Biol. Clifton Nj 2004; 246:391-411.

4. Markowitz D, Hesdorffer C, Ward M, Goff S, Bank A. Retroviral gene transfer using safe and efficient packaging cell lines. Ann. N. Y. Acad. Sci. 1990; 612:407-414.

5. Grignani F, Kinsella T, Mencarelli A, Valtieri M, Riganelli D, Grignani F, Lanfrancone L, Peschle C, Nolan G P, Pelicci P G. High-efficiency gene transfer and selection of human hematopoietic progenitor cells with a hybrid EBV/retroviral vector expressing the green fluorescence protein. Cancer Res. 1998; 58:14-19.

6. Naldini L, Blomer U, Gallay P, Ory D, Mulligan R, Gage F H, Verma I M, Trono D. In vivo gene delivery and stable transduction of nondividing cells by a lentiviral vector. Science 1996; 272:263-267.

7. Wu Y, Tapia P H, Fisher G W, Simons P C, Strouse J J, Foutz T, Waggoner A S, Jarvik J, Sklar L A. Discovery of regulators of receptor internalization with high-throughput flow cytometry. Mol. Pharmacol. 2012; 82:645-657.

8. Heim R, Cubitt A B, Tsien R Y. Improved green fluorescence. Nature 1995; 373:663-664.

9. Fiering S N, Roederer M, Nolan G P, Micklem D R, Parks D R, Herzenberg L A. Improved FACS-Gal: Flow cytometric analysis and sorting of viable eukaryotic cells expressing reporter gene constructs. Cytometry 1991; 12:291-301.

10. Schulz K R, Danna E A, Krutzik P 0, Nolan G P. Single-cell phospho-protein analysis by flow cytometry. Curr. Protoc. Immunol. Ed. John E Coligan Al 2007; Chapter 8:Unit 8.17.

11. Piatkevich K D, Malashkevich V N, Almo S C, Verkhusha V V. Engineering ESPT Pathways Based on Structural Analysis of LSSmKate Red Fluorescent Proteins with Large Stokes Shift. J. Am. Chem. Soc. 2010; 132:10762-10770.

12. Piatkevich K D, Hulit J, Subach O M, Wu B, Abdulla A, Segall J E, Verkhusha V V. Monomeric red fluorescent proteins with a large Stokes shift. Proc. Natl. Acad. Sci. 2010. Available at: http://www.pnas.org/content/early/2010/03/02/0914365107. Accessed May 14, 2013.

13. Tsien R Y. Nobel lecture: constructing and exploiting the fluorescent protein paintbox. Integr. Biol. Quant. Biosci. Nano Macro 2010; 2:77-93.

14. Shaner N C, Lin M Z, McKeown M R, Steinbach P A, Hazelwood K L, Davidson M W, Tsien R Y. Improving the photostability of bright monomeric orange and red fluorescent proteins. Nat. Methods 2008; 5:545-551.

15. Gurskaya N G, Verkhusha V V, Shcheglov A S, Staroverov D B, Chepurnykh T V, Fradkov A F, Lukyanov S, Lukyanov K A. Engineering of a monomeric green-to-red photoactivatable fluorescent protein induced by blue light. Nat. Biotechnol. 2006; 24:461-465.

16. Subach F V, Subach O M, Gundorov I S, Morozova K S, Piatkevich K D, Cuervo A M, Verkhusha V V. Monomeric fluorescent timers that change color from blue to red report on cellular trafficking. Nat. Chem. Biol. 2009; 5:118-126.

17. Buranda T, Wu Y, Sklar L A. Quantum dots for quantitative flow cytometry. Methods Mol. Biol. Clifton Nj 2011; 699:67-84.

18. Shaner N C, Campbell R E, Steinbach P A, Giepmans B N G, Palmer A E, Tsien R Y. Improved monomeric red, orange and yellow fluorescent proteins derived from Discosoma sp. red fluorescent protein. Nat. Biotechnol. 2004; 22:1567-1572.

19. Grant G D, Gamsby J, Martyanov V, Brooks L 3rd, George L K, Mahoney J M, Loros J J, Dunlap J C, Whitfield M L. Live-cell monitoring of periodic gene expression in synchronous human cells identifies Forkhead genes involved in cell cycle control. Mol. Biol. Cell 2012; 23:3079-3093.

20. Krutzik P O, Nolan G P. Intracellular phospho-protein staining techniques for flow cytometry: monitoring single cell signaling events. Cytom. Part J. Int. Soc. Anal. Cytol. 2003; 55:61-70.

21. Lu R, Neff N F, Quake S R, Weissman I L. Tracking single hematopoietic stem cells in vivo using high-throughput sequencing in conjunction with viral genetic barcoding. Nat. Biotechnol. 2011; 29:928-933.

22. Krutzik P O, Nolan G P. Fluorescent cell barcoding in flow cytometry allows high-throughput drug screening and signaling profiling. Nat. Methods 2006; 3:361-368.

23. Edwards B S, Ivnitski-Steele I, Young S M, Salas V M, Sklar L A. High-throughput cytotoxicity screening by propidium iodide staining. Curr. Protoc. Cytom. Editor. Board J Paul Robinson Manag. Ed. Al 2007; Chapter 9:Unit9.24.

24. Violin J D, Zhang J, Tsien R Y, Newton A C. A genetically encoded fluorescent reporter reveals oscillatory phosphorylation by protein kinase C. J. Cell Biol. 2003; 161:899-909.

25. Ashcroft R G, Lopez P A. Commercial high speed machines open new opportunities in high throughput flow cytometry (HTFC). J. Immunol. Methods 2000; 243:13-24.

26. Durick K, Negulescu P. Cellular biosensors for drug discovery. Biosens. Bioelectron. 2001; 16:587-592.

27. Edwards B S, Young S M, Saunders M J, Bologa C, Oprea T I, Ye R D, Prossnitz E R, Graves S W, Sklar L A. High-throughput flow cytometry for drug discovery. Expert Opin. Drug Discov. 2007; 2:685-696.

28. Sklar L A, Carter M B, Edwards B S. Flow cytometry for drug discovery, receptor pharmacology and high-throughput screening. Curr. Opin. Pharmacol. 2007; 7:527-534.

29. Curpan R F, Simons P C, Zhai D, Young S M, Carter M B, Bologa C G, Oprea T I, Satterthwait A C, Reed J C, Edwards B S, Sklar L A. High-throughput screen for the chemical inhibitors of antiapoptotic bcl-2 family proteins by multiplex flow cytometry. Assay Drug Dev. Technol. 2011; 9:465-474.

30. Perez O D, Nolan G P. Simultaneous measurement of multiple active kinase states using polychromatic flow cytometry. Nat. Biotechnol. 2002; 20:155-162.

31. Edwards B S, Oprea T, Prossnitz E R, Sklar L A. Flow cytometry for high-throughput, high-content screening. Curr. Opin. Chem. Biol. 2004; 8:392-398.

32. Vignali D A. Multiplexed particle-based flow cytometric assays. J. Immunol. Methods 2000; 243:243-255.

33. Krutzik P O, Clutter M R, Trejo A, Nolan G P. Fluorescent Cell Barcoding for Multiplex Flow Cytometry. Curr. Protoc. Cytom. Editor. Board J Paul Robinson Manag. Ed. Al 2011; CHAPTER:Unit-6.31.

34. Edwards B S, Sklar L A. Plug flow cytometry. Curr. Protoc. Cytom. Editor. Board J Paul Robinson Manag. Ed. Al 2001; Chapter 1:Unit 1.17.

35. Edwards B S, Young S M, Ivnitsky-Steele I, Ye R D, Prossnitz E R, Sklar L A. High-content screening: flow cytometry analysis. Methods Mol. Biol. Clifton Nj 2009; 486:151-165.

36. Haynes M K, Strouse J J, Waller A, Leitao A, Curpan R F, Bologa C, Oprea T I, Prossnitz E R, Edwards B S, Sklar L A, Thompson T A. Detection of intracellular granularity induction in prostate cancer cell lines by small molecules using the HyperCyt high-throughput flow cytometry system. J. Biomol. Screen. 2009; 14:596-609.

37. Bradford J A, Buller G, Suter M, Ignatius M, Beechem J M. Fluorescence-intensity multiplexing: simultaneous seven-marker, two-color immunophenotyping using flow cytometry. Cytom. Part J. Int. Soc. Anal. Cytol. 2004; 61:142-152.

38. Beske O E, Goldbard S. High-throughput cell analysis using multiplexed array technologies. Drug Discov. Today 2002; 7:S131-S135.

39. Surviladze Z, Young S M, Sklar L A. High-throughput flow cytometry bead-based multiplex assay for identification of Rho GTPase inhibitors. Methods Mol. Biol. Clifton Nj 2012; 827:253-270.

40. Chen J, Young S M, Allen C, Seeber A, Pèli-Gulli M-P, Panchaud N, Waller A, Ursu O, Yao T, Golden J E, Strouse J J, Carter M B, Kang H, Bologa C G, Foutz T D, Edwards B S, Peterson B R, Aube J, Werner-Washburne M, Loewith R J, De Virgilio C, Sklar L A. Identification of a small molecule yeast TORC1 inhibitor with a multiplex screen based on flow cytometry. Acs Chem. Biol. 2012; 7:715-722.

41. Quinonez R, Sutton R E. Lentiviral vectors for gene delivery into cells. Dna Cell Biol. 2002; 21:937-951.

42. Woods N B, Mikkola H, Nilsson E, Olsson K, Trono D, Karlsson S. Lentiviral-mediated gene transfer into haematopoietic stem cells. J. Intern. Med. 2001; 249:339-343.

43. Blomer U, Naldini L, Kafri T, Trono D, Verma I M, Gage F H. Highly efficient and sustained gene transfer in adult neurons with a lentivirus vector. J. Virol. 1997; 71:6641-6649.

44. Nguyen T H, Oberholzer J, Birraux J, Majno P, Morel P, Trono D. Highly efficient lentiviral vector-mediated transduction of nondividing, fully reimplantable primary hepatocytes. Mol. Ther. J. Am. Soc. Gene Ther. 2002; 6:199-209.

45. Engelman A, Mizuuchi K, Craigie R. HIV-1 DNA integration: Mechanism of viral DNA cleavage and DNA strand transfer. Cell 1991; 67:1211-1221.

46. Stolp Z D, Stotland A, Diaz S, Hilton B J, Burford W, Wolkowicz R. A Novel Two-Tag System for Monitoring Transport and Cleavage through the Classical Secretory Pathway—Adaptation to HIV Envelope Processing. Plos One 2013; 8:e68835.

47. Hilton B J, Wolkowicz R. An assay to monitor HIV-1 protease activity for the identification of novel inhibitors in T-cells. Plos One 2010; 5:e10940.

48. Gubin A N, Reddy B, Njoroge J M, Miller J L. Long-Term, Stable Expression of Green Fluorescent Protein in Mammalian Cells. Biochem. Biophys. Res. Commun 1997; 236:347-350.

49. Condreay J P, Witherspoon S M, Clay W C, Kost T A. Transient and stable gene expression in mammalian cells transduced with a recombinant baculovirus vector. Proc. Natl. Acad. Sci. 1999; 96:127-132.

50. Levy J P, Muldoon R R, Zolotukhin S, Link C J. Retroviral transfer and expression of a humanized, red-shifted green fluorescent protein gene into human tumor cells. Nat. Biotechnol. 1996; 14:610-614.

51. Limon A, Briones J, Puig T, Carmona M, Fornas O, Cancelas J A, Nadal M, Garcia J, Rueda F, Barquinero J. High-Titer Retroviral Vectors Containing the Enhanced Green Fluorescent Protein Gene for Efficient Expression in Hematopoietic Cells. Blood 1997; 90:3316-3321.

52. Shaner N C, Steinbach P A, Tsien R Y. A guide to choosing fluorescent proteins. Nat. Methods 2005; 2:905-909.

### REFERENCES FOR FLUORESCENT PROTEINS

1. Matz, M. V., et al., Fluorescent proteins from nonbioluminescent Anthozoa species. Nat Biotechnol, 1999. 17: p. 969-973.

2. Griesbeck, O., et al., Reducing the environmental sensitivity of yellow fluorescent protein. Mechanism and applications. J Biol Chem, 2001. 276(31): p. 29188-94.

3. Campbell, R. E., et al., A monomeric red fluorescent protein. Proc Natl Acad Sci USA, 2002. 99(12): p. 7877-82.

4. Bevis, B. J. and B. S. Glick, Rapidly maturing variants of the Discosoma red fluorescent protein (DsRed). Nat Biotechnol, 2002. 20(1): p. 83-7.

5. Gurskaya, N. G., et al., GFP-like chromoproteins as a source of far-red fluorescent proteins. FEBS Lett, 2001. 507(1): p. 16-20.

6. Lukyanov, K. A., et al., Natural animal coloration can be determined by a nonfluorescent green fluorescent protein homolog. J Biol Chem, 2000. 275(34): p. 25879-82.

7. Wiedenmann, J., et al., A far-red fluorescent protein with fast maturation and reduced oligomerization tendency from Entacmaea quadricolor (Anthozoa, Actinaria). Proc Natl Acad Sci USA, 2002. 99(18): p. 11646-51.

8. Cubitt, A. B., L. A. Woollenweber, and R. Heim, Understanding structure-function relationships in the Aequorea victoria green fluorescent protein. Meth Cell Biol, 1999. 58: p. 19-30.

9. Nagai, T., et al., A variant of yellow fluorescent protein with fast and efficient maturation for cell-biological applications. Nat Biotechnol, 2002. 20(1): p. 87-90.

10. Patterson, G. H. and J. Lippincott-Schwartz, A Photoactivatable GFP for Selective Photolabeling of Proteins and Cells. Science, 2002. 297(5588): p. 1873-7.

11. Karasawa, S. et al., A green-emitting fluorescent protein from Galaxeidae coral and its monomeric version for use in fluorescent labeling. J Biol Chem, 2003. 278(36):p. 34167-71.

12. Zapata-Hommer O. and Griesbeck O., Efficiently folding and circularly permuted variants of the Sapphire mutant of GFP. BMC Biotechnol, 2003. 3(5).

13. Rizzo, M. A., et al., An improved cyan fluorescent protein variant useful for FRET. Nat Biotechnol, 2004. 22(4):p. 445-449.

14. Karasawa, S., et al., Cyan-emitting and orange-emitting fluorescent proteins as a donor/acceptor pair for fluorescence resonance energy transfer. Biochem J, 2004. 381:p. 307-312.

15. Wang, L. et al., Evolution of new non-antibody proteins via iterative somatic hypermutation. Proc Natl Acad Sci, 2004. 101(48):p. 16745-16749.

16. Shaner, N. C. et al., Improved monomeric red, orange, and yellow fluorescent proteins derived from Discosoma sp. red fluorescent protein. Nat Biotechnol, 2004.

17. Chudakov, D. M. et al., Photoswitchable cyan fluorescent protein for protein tracking. Nat Biotechnol 2004. 22(11):p. 1435-1439.

18. Wiedenmann, J. et al., EosFP, a fluorescent marker protein with UV-inducible green-to-red fluorescence conversion. Proc Natl Acad Sci 2004. 101(45): p.15905-15910.

19. Ando, R. et al., Regulated fast nucleocytoplasmic shuttling observed by reversible protein highlighting. Science 2004. 306: p. 1370-1373.

20. Merzlyak, E. M. et al., Bright monomeric red fluorescent protein with an extended fluorescence lifetime. Nat. Methods 2007.4(7): p. 555-557.

21. Shcherbo, D. et al., Bright far-red fluorescent protein for whole-body imaging. Nat. Methods 2007. 4(9): p. 741-746.

22. Kogure, T. et al., A fluorescent variant of a protein from the stony coral Montipora facilitates dual-color single-laser fluorescence cross-correlation spectroscopy. Nat. Biotechnol. 2006. 24(5): p. 557-581.

23. Tsutsui, H. et al. Semi-rational engineering of a coral fluorescent protein into an efficient highlighter. EMBO Rep. 2005. 6(3): p. 233-238.

24. Ai H W, et al., Directed evolution of a monomeric, bright and photostable version of Clavularia cyan fluorescent protein: structural characterization and applications in fluorescence imaging. Biochem J. 2006. 400(3): p. 531-540.

25. Shkrob M A, et al., Far-red fluorescent proteins evolved from a blue chromoprotein from Actinia equina. Biochem J. 2005. 392(Pt 3): p. 649-654.

26. Tsutsui H, et al., Improving membrane voltage measurements using FRET with new fluorescent proteins. Nat. Methods 2008. 5(8): p. 683-685.

27. Subach O M et al., Conversion of Red Fluorescent Protein into a Bright Blue Probe. Chemistry & Biology. 2008. 15(10): p. 1116-1124.

28. Mena, M A et al., Blue fluorescent proteins with enhanced brightness and photostability from a structurally targeted library. Nat Biotech. 2008. 24(12):p. 1569-1571.

29. Ai, H, et al., Exploration of new chromophore structures leads to the identification of improved blue fluorescent proteins. Biochemistry 2007, 46: p. 5904-5910.

30. Morozova K S et al., Far-red fluorescent protein excitable with red lasers for flow cytometry and superresolution STED nanoscopy. Biophys. J. 2010, 99: p. L13-15.

31. Shu X et al., Mammalian Expression of Infrared Fluorescent Proteins Engineered from a Bacterial Phytochrome. Science 2009, 324: p804-807.

32. Piatkevich K D et al., Monomeric red fluorescent proteins with a large Stokes shift. PNAS 2010, 107: p. 5369-5374.

33. Shaner N C et al., Improving the photostability of bright monomeric orange and red fluorescent proteins. Nat. Meth. 2008, 5: p.545-551.

34. Lin M Z et al., Autofluorescent proteins with excitation in the optical window for intravital imaging in mammals. Chem. Biol. 2009, 16: p. 1169-1179.

35. Kredel S. et al., mRuby, a bright monomeric red fluorescent protein for labeling of subcellular structures. PLOS One 2009, 4: e4391.

36. Goedhart J. et al., Bright cyan fluorescent protein variants identified by fluorescence lifetime screening. Nat. Meth. 2010, 7: p. 137-141.

37. Tomosugi W. et al., An ultramarine fluorescent protein with increased photostability and pH insensitivity. Nat. Meth. 2009, 5: p. 351-353.

38. Pedelacq J-D. et al., Engineering and characterization of a superfolder green fluorescent protein. Nat. Biotech. 2006, 24: p. 79-88.

39. Kremers, G-J. et al., Cyan and Yellow Super Fluorescent Proteins with Improved Brightness, Protein Folding, and FRET Forster Radius. Biochem. 2006, 45: p. 6570-6580.

40. Subach, F V. et al., Bright Monomeric Photoactivatable Red Fluorescent Protein for Two-Color Super-Resolution sptPALM of Live Cells. JACS 2010, 132: p. 6481-6491.

41. Subach, F V. et al., Photoactivatable mCherry for high-resolution two-color fluorescence microscopy. Nat. Meth. 2009, 6: p. 153-159.

42. McKinney, S A. et al., A bright and photostable photoconvertible fluorescent protein. Nat Meth. 2009, 6: p. 131-133.

43. Filonov, G S. et al., Bright and stable near-infrared fluorescent protein for in vivo imaging. Nat. Biotech. 2011, 29: p. 757-761.

44. Subach, O M. et al., A photoswitchable orange-to-far-red fluorescent protein, PSmOrange. Nat. Meth. 2011, 8: p. 771-777.

45. Zhang, M. et al., Rational design of true monomeric and bright photoactivatable fluorescent proteins. Nat. Meth. 2012, 9: p. 727-729.

46. Lam, A J. et al., Improving FRET dynamic range with bright green and red fluorescent proteins. Nat Meth. Sep. 9, 2012 doi: 10.1038/nmeth.2171.

47. Goedhart, J. et al., Structure-guided evolution of cyan fluorescent proteins towards a quantum yield of 93%. Nat Commun. Mar. 20, 2012; 3:751. doi: 10.1038/ncomms1738.

48. Subach, O M. et al, An enhanced monomeric blue fluorescent protein with the high chemical stability of the chromophore. PLoS One. 2011; 6(12):e28674.

49. Shaner, N C. et al, A bright monomeric green fluorescent protein derived from Branchiostoma lanceolatum. Nat. Meth. Mar. 24, 2013 doi: 10.1038/nmeth.2413

50. Yang, J. et al, mBeRFP, an Improved Large Stokes Shift Red Fluorescent Protein. PLOS One 2013; 8(6):e64849.

A number of embodiments of the invention have been described. Nevertheless, it will be understood that various modifications may be made without departing from the spirit and scope of the invention. Accordingly, other embodiments are within the scope of the following claims.

1. A method for tagging, uniquely identifying or genetically barcoding a cell, a population of cells, or a culture of cells, comprising:
(a) providing a cell, a population of cells or a culture of cells;
(b) providing at least one nucleic acid encoding a readable or detectable moiety, wherein the at least one nucleic acid is contained within a vehicle, plasmid, vector or recombinant virus, or equivalent thereof, capable of stably transfecting, transducing, infecting or implanting in the cell; and
(c) transferring, transfecting, transducing, infecting or implanting the at least one readable or detectable moiety-encoding nucleic acid into the cell or culture of cells, thereby stably transfecting, transducing, infecting or implanting in the cell the vehicle, plasmid, vector or recombinant virus, or equivalent thereof.

2. The method of claim 1, further comprising culturing the cell or culture of cells and expressing the readable or detectable moiety, or culturing the cell or culture of cells under conditions in which the readable or detectable moiety is expressed.

3. The method of claim 1, further comprising isolating, counting or characterizing the tagged, uniquely identified or genetically barcoded cell or a population of cells.

4. The method of claim 3, wherein the tagged, uniquely identified or genetically barcoded cell or a population of cells is isolated, counted or characterized using a flow cytometry or a fluorescent activated cell sorting (FACS), or equivalent.

5. The method of claim 1, wherein the transferring, transfecting, transducing, infecting or implanting is in vitro, ex vivo or in vivo, and optionally the cell or the population of cells is in vivo.

6. The method of any of claims 1, further comprising identifying or counting the number of readable or detectable moiety expressing cells.

7. The method of claim 1, wherein the readable or detectable moiety comprises a fluorescent protein, or the nucleic acid encodes a protein detectable by a fluorescent, a luminescent, a colorimetric or an equivalent detection assay.

8. The method of claim 7, wherein the fluorescent protein comprises a member of the group consisting of: a green fluorescent protein (GFP), an mCherry protein, a td Tomato protein, an E2 Crimson protein, a Cerulean protein, an mBanana protein and a combination thereof.

9. The method of any of claim 1, wherein: at least two, three, four or five or more different readable or detectable moieties are transferred into the cell or culture of cells; or, at least two, three, four or five or more different nucleic acids encoding different or distinguishable readable or detectable moieties are stably transfected, transduced, infected or implanted in the cell.

10. The method of claim 9, wherein each of the at least two, three, four or five or more different readable or detectable moieties has a spectrum range that does not overlap, or does not significantly overlap, with any of the other readable or detectable moieties.

11. The method of claim 10, wherein clonal cell populations carrying individual readable or detectable moieties, or fluorescent proteins, are selected based on expression at different intensities, and optionally the expression level is such that the value of the mean fluorescence intensity of each population is one log scale apart to make them distinguishable.

12. The method of claim 1, wherein the at least one nucleic acid is operably linked to an inducible or a constitutively active transcriptional activator or promoter; or, if more than one readable or detectable moieties are used, each type of nucleic acid is operably linked to its own transcriptional activator or promoter (each class of readable or detectable moieties is operatively linked to a different transcriptional activator or promoter).

13. The method of claim 1, wherein the vehicle, plasmid, vector or recombinant virus, or equivalent thereof, is capable of stably transfecting the cell is or comprises:
a recombinant retrovirus or a lentiviral recombinant virus, or
a Vesicular Stomatitis Virus, or a Vesicular Stomatitis Virus Envelope glycoprotein vector, or equivalent thereof.

14. The method of claim 1, further comprising use of antibody staining to count or identify a cell or a cell population or a phenotype.

15. The method of claim 1, wherein the cells are mammalian cells, animal cells, or human cells, or are cells derived from cell lines or stable cell cultures.

16. A cell or a population of cells tagged, uniquely identified or genetically barcoded using a method of claim 1.

17. A non-human animal or non-human mammal comprising a cell or a population of cells tagged, uniquely identified or genetically barcoded using a method of claim 1.

18. The method of claim 1, wherein the vehicle, plasmid, vector or recombinant virus, or equivalent thereof, is stably replicated by the cell during mitosis as an autonomous structure.

19. The method of claim 1, wherein the vehicle, plasmid, vector or recombinant virus, or equivalent thereof, is incorporated within the cell's genome.

20. The method of claim 13, wherein the vehicle, plasmid, vector or recombinant virus, or equivalent thereof, is capable of stably transfecting the cell is or comprises a vehicle, plasmid, vector or recombinant virus, or equivalent thereof, capable of targeting a particular cell type or cell phenotype.
